(19) United States
(12) Patent Application Publication
(10) Pub. No.: US 2026/0054027 A1
(43) Pub. Date: Feb. 26, 2026

(54) SYSTEMS, METHODS, AND APPARATUS FOR PRODUCING NITRIC OXIDE

(71) Applicant: VERO Biotech Inc., Atlanta, GA (US)

(72) Inventors: Christopher Varga, Atlanta, GA (US); Aaron Roebuck, Atlanta, GA (US)

(21) Appl. No.: 19/303,624

(22) Filed: Aug. 19, 2025

Related U.S. Application Data
(60) Provisional application No. 63/685,413, filed on Aug. 21, 2024.

(51) Int. Cl. A61M 16/12; A61M 1/14; A61M 16/08; C01B 21/24

(52) U.S. Cl. CPC A61M 16/122; A61M 1/14; A61M 16/085; C01B 21/24; A61M 2202/0275; A61M 2205/3303

(57) ABSTRACT

Disclosed herein is a system and method comprising an apparatus for delivering a therapeutic amount of nitric oxide to a subject, the apparatus comprising at least one air inlet; a first receptacle configured to host a first source of nitric oxide; a second receptacle configured to host a second source of nitric oxide; a control unit configured to communicate with the first receptacle and the second receptacle and to selectively couple the at least one air inlet to at least one of the first receptacle or the second receptacle to deliver the nitric oxide from the first or the second source; a first outlet coupled to the first receptacle to deliver nitric oxide; and a second outlet coupled to the second receptacle to deliver nitric oxide. The first source can be provided to a ventilator and the second source can be provided to an ECMO device.

### CROSS-REFERENCE TO RELATED APPLICATIONS

This application claims the benefit of U.S. Provisional Application No. 63/685,413, filed Aug. 21, 2024, the content of which is incorporated herein by reference in its entirety.

### FIELD

Some aspects described herein relate to a medical device and, more particularly, to systems and methods for producing and delivering a gas that includes nitric oxide.

### BACKGROUND

Some aspects described herein relate to the production of nitric oxide (NO), which is then typically delivered to a patient in a medical setting.

Nitric oxide is a vasodilator indicated to improve oxygenation and reduce the need for extracorporeal membrane oxygenation, particularly in term and near-term neonates with hypoxic respiratory failure associated with clinical or echocardiographic evidence of pulmonary hypertension in conjunction with ventilatory support. Low concentrations of inhaled nitric oxide can also prevent, reverse, or limit the progression of disorders, which can include, but are not limited to, acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery acute pulmonary hypertension, persistent pulmonary hypertension of a newborn, perinatal aspiration syndrome, hyaline membrane disease, acute pulmonary thromboembolism, heparin-protamine reactions, sepsis, asthma and status asthmaticus or hypoxia. Nitric oxide can also be used to treat chronic pulmonary hypertension, bronchopulmonary dysplasia, chronic pulmonary thromboembolism, idiopathic or primary pulmonary hypertension, and chronic hypoxia.

Inhaled nitric oxide therapy typically involves delivering nitric oxide in concentrations ranging from parts per billion to parts per million within a breathing gas, generally composed of air or oxygen-enriched air. This breathing gas may contain other components, such as anesthetic agents, nebulized liquids, or other gaseous components, and it is typically conveyed to a patient using either a mechanical or manual ventilation device. In some inhaled nitric oxide delivery systems, nitric oxide is provided within pressurized tanks, whereas in other systems, it may be generated on demand within the delivery system itself. One such system is described in U.S. Pat. No. 11,744,978, the content of which is incorporated herein in its entirety. In this approach, nitric oxide is produced through a chemical reaction between NO₂ gas and an antioxidant, where the NO₂ gas is generated via a phase change of liquid N₂O₄. In such systems, liquid N₂O₄ is typically housed in a pressure vessel with components required for reaction control (e.g., heating and cooling components), reactant mixing, and measurement, all of which are co-located with the reactants themselves. Although this is an effective approach, there is a need for a system wherein the reactants required to create nitric oxide gas for a patient are housed within a simple one-time-use component, and the components that are required to initiate, contain, measure, and control the reaction reside in a location where they can be used many times. This creates the need for novel packaging, geometries, and orientations of reactants, as well as novel loading, activation, and ejection mechanisms.

One particular use for the nitric oxide delivery is for life support where a patient with compromised lungs. While mechanical ventilation is the most common form of life support, it can only provide gas flow into and out of the lungs for the exchange of oxygen and carbon dioxide with the blood. Extracorporeal Membrane Oxygen (ECMO) treatment is a specialized type of cardiopulmonary bypass (CPB) and a form of life support that can also be utilized on patients whose lungs and hearts are compromised to exchange oxygen and carbon dioxide directly with the blood. ECMO pumps blood out of the body and sends it through a membrane oxygenator (artificial lung) that removes carbon dioxide and adds oxygen to the blood. This blood is then rewarmed and returned to the body, allowing the lungs and heart to rest and/or heal. Common conditions that ECMO treats include Acute Respiratory Distress Syndrome (ARDS), heart trauma, pulmonary embolism, infants and newborns born prematurely with lung and heart problems, and it is also used in surgeries and transplants, including recovery after surgery. During ECMO treatment, a patient often remains on mechanical ventilation to keep the lungs mildly ventilated and open as well as to supplement ECMO blood flow rates. While the addition of nitric oxide gas to mechanical ventilation gas is common, it has also been found to be beneficial to add nitric oxide gas to the ECMO treatment circuit. The ECMO treatment circuit consists of a gas flow called “sweep” gas that is generally composed of air and oxygen, but may also contain other gases or anesthetic agents. This gas is directed to a membrane oxygenator to remove carbon dioxide and transfer oxygen to the blood that is passed through the device. Adding nitric oxide to this sweep gas can be challenging as the sweep gas flow rates required for small patients are typically very low, requiring both high-precision nitric oxide dosing as well as low gas sampling flow rates. Furthermore, adding nitric oxide to this sweep gas during ECMO treatment while also adding nitric oxide into the mechanical ventilation circuit generally requires multiple nitric oxide delivery systems because the dosing may be different for each injection location (i.e., ventilation circuit and ECMO circuit). Control functions and delivery mechanisms for state-of-the-art nitric oxide delivery systems are not directed to either ECMO-specific scenarios nor to dual-delivery scenarios with independent dose, feedback control, and the ability to quantify the total nitric oxide being dosed through both the ventilator and the ECMO circuit. Accordingly, there is a need for a user to have an apparatus that is capable of both ECMO-specific nitric oxide delivery as well as dual-dosing to an ECMO system and a mechanical ventilation system simultaneously with independent dose control, feedback, and quantification of total nitric oxide delivery to the patient.

This need and all other needs are at least partially addressed by this disclosure.

### SUMMARY

The present disclosure is directed to a single-use container for forming a therapeutic amount of nitric oxide to be delivered by an apparatus to a subject, wherein the single-use container comprises: a housing defined by a proximal edge and a distal edge and comprising: a first chamber comprising N₂O₄, wherein the first chamber is sealed; a second chamber comprising an antioxidant material; and wherein the first chamber and the second chamber are positioned relative to each other such that when the single-use container is inserted into the apparatus and is activated, the first chamber is unsealed to allow the N₂O₄ to be in fluid communication with the antioxidant material in the second chamber to produce nitric oxide.

In still further aspects, the disclosure is directed to an apparatus comprising: an anvil positioned within a receptacle of the apparatus, wherein the receptacle defines a space configured to receive the single-use container of any of the examples herein, wherein the anvil is engageable with at least the proximal edge of the single-use container.

Still further disclosed herein is a system comprising: any of the disclosed herein single-use containers and/or any of the disclosed herein apparatuses.

Additional advantages will be set forth in part in the description that follows, and in part will be obvious from the description or can be learned by practice of the aspects described below. The advantages described below will be realized and attained by means of the chemical compositions, methods, and combinations thereof, particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1 is a schematic of an exemplary system according to one aspect of the disclosure.

FIG. 2 is a schematic of an exemplary system according to one aspect of the disclosure.

FIG. 3 is a schematic of an exemplary system according to one aspect of the disclosure.

FIG. 4 is a schematic of an exemplary system according to one aspect of the disclosure.

FIG. 5 is a schematic of an exemplary system according to one aspect of the disclosure.

FIG. 6 is a schematic of an exemplary system according to one aspect of the disclosure.

FIGS. 7A-7I show various exemplary systems according to one aspect of the disclosure.

FIG. 8 is a schematic of an exemplary system according to one aspect of the disclosure.

FIG. 9 shows a side and a top view schematic of an exemplary single-use container before it is engaged with an exemplary anvil of an apparatus according to one aspect of the disclosure.

FIGS. 10A-10D are side-view schematics of an exemplary single-use container engaged with an exemplary anvil of an apparatus at different steps of delivering nitric oxide according to one aspect of the disclosure. FIG. 10E shows a top view schematic of an exemplary single-use container engaged with an exemplary anvil of an apparatus after the deactivation of the single-use container. FIG. 10F shows a side view schematics after disengaging the single-use container from the anvil and removal of the single-use container from the apparatus.

FIGS. 11A-11J show various views of the engagement of the single-use container within the apparatus.

FIGS. 12A-12F show various steps of operation of the exemplary system according to some aspects of the disclosure.

FIGS. 13A-13B depict a schematic of an exemplary single-use container according to some aspects of the disclosure.

FIGS. 14A-14C depict a closer view of an exemplary part of the exemplary single-use container of FIGS. 13A-13B.

FIG. 15 depicts a closer view of an exemplary part of the exemplary single-use container of FIGS. 13A-13B.

FIG. 16 depicts a closer view of an exemplary part of the exemplary single-use container of FIGS. 13A-13B.

FIG. 17 depicts a closer view of an exemplary part of the exemplary single-use container of FIGS. 13A-13B.

FIG. 18 depicts various steps of operation of the exemplary single-use container of FIGS. 13A-13B.

FIG. 19 depicts a disengagement step of the exemplary single-use container of FIGS. 13A-13B at the end of the operation.

FIGS. 20A-20C depict an exemplary flow-directing unit connected to the first chamber according to one aspect of the disclosure. FIGS. 21A-21B depict an exemplary single-use using the flow-directing unit and the first chamber shown in FIGS. 20A-20C according to one aspect of the disclosure.

FIGS. 22A-22C depict an exemplary flow-directing unit connected to the first chamber according to one aspect of the disclosure.

FIGS. 23A-23C depict an exemplary single-use container comprising the flow-directing unit and the first chamber shown in FIGS. 22A-22C according to one aspect of the disclosure.

FIG. 24 depicts exemplary needle configurations.

FIGS. 25A-25C depict an exemplary flow-directing unit connected to the first chamber according to one aspect of the disclosure.

FIGS. 26A-26E depict an exemplary single-use using the flow-directing unit and the first chamber shown in FIGS. 25A-25C according to one aspect of the disclosure.

FIG. 27 depicts an exemplary receptacle containing the single-use container of FIGS. 25A-26E and an exemplary anvil in one aspect of the disclosure.

FIG. 28 depicts exemplary steps of using the single-use container in the apparatus according to one aspect of the disclosure.

FIGS. 29A-29D depict an exemplary flow-directing unit connected to the first chamber according to one aspect of the disclosure.

FIGS. 30A-30D depict an exemplary receptacle containing the single-use container of FIGS. 29A-29D and an exemplary anvil in one aspect of the disclosure.

FIG. 31 depicts an exemplary receptacle containing the single-use container of FIGS. 29A-30D and an exemplary anvil in one aspect of the disclosure

FIGS. 32A-32D depict an exemplary flow-directing unit connected to the first chamber according to one aspect of the disclosure.

FIGS. 33A-33F depict various views of the exemplary single-use container according to one aspect of the disclosure.

FIGS. 34A-34G depict exemplary views of an exemplary valve.

FIG. 35 depicts a photograph of an exemplary single-use container.

FIGS. 36A-36F depict an assembled view (FIG. 36A) and exploded views (FIGS. 36B-36F) of the single-use container according to some aspects of the disclosure.

FIGS. 37A-37B depict a first diagonal cutaway view (FIG. 37A) and a second diagonal cutaway view (FIG. 37B) of the single-use container according to some aspects of the disclosure

FIG. 38 shows an exemplary apparatus according to some aspects of this disclosure and provided as an alternative to the apparatus in FIGS. 7A-I with a second outlet for nitric oxide.

FIG. 39 shows an exemplary system according to some aspects of this disclosure wherein a first source of nitric oxide is provided to a ventilator and a second source of nitric oxide is provided to an ECMO device.

FIG. 40 shows an exemplary system according to some aspects of this disclosure wherein a first source of nitric oxide and a second source of nitric oxide are provided to an ECMO device.

FIG. 41 shows an exemplary system according to some aspects of this disclosure wherein a first source of nitric oxide and a second source of nitric oxide are provided to a ventilator.

The accompanying figures, which are incorporated in and constitute a part of this specification, illustrate several aspects described below.

### DETAILED DESCRIPTION

The present invention can be understood more readily by reference to the following detailed description, examples, drawings, and claims, and their previous and following description. However, before the present articles, systems, and/or methods are disclosed and described, it is to be understood that this invention is not limited to the specific or exemplary aspects of articles, systems, and/or methods disclosed unless otherwise specified, as such can, of course, vary. It is also to be understood that the terminology used herein is for the purpose of describing particular aspects only and is not intended to be limiting.

The following description of the invention is provided as an enabling teaching of the invention in its best, currently known aspect. To this end, those skilled in the relevant art will recognize and appreciate that many changes can be made to the various aspects of the invention described herein while still obtaining the beneficial results of the present invention. It will also be apparent that some of the desired benefits of the present invention can be obtained by selecting some of the features of the present invention without utilizing other features. Accordingly, those of ordinary skill in the pertinent art will recognize that many modifications and adaptations to the present invention are possible and may even be desirable in certain circumstances and are a part of the present invention. Thus, the following description is again provided as illustrative of the principles of the present invention and not in limitation thereof.

### Definitions

As used herein, the terms “optional” or “optionally” mean that the subsequently described event or circumstance can or cannot occur and that the description includes instances where said event or circumstance occurs and instances where it does not.

It is appreciated that certain features of the disclosure, which are, for clarity, described in the context of separate aspects, can also be provided in combination in a single aspect. Conversely, various features of the disclosure, which are, for brevity, described in the context of a single aspect, can also be provided separately or in any suitable subcombination.

As used in the description and the appended claims, the singular forms “a,” “an,” and “the” include plural referents unless the context clearly dictates otherwise. Thus, for example, a reference to “a single-use unit” includes not only one but also two or more such units, and a reference to “an apparatus” includes not only one but also two or more such apparatuses and the like.

Throughout the description and claims of this specification, the word “comprise” and other forms of the word, such as “comprising” and “comprises,” are open, non-limiting terms and mean “including but not limited to,” and are not intended to exclude, for example, other additives, segments, integers, or steps. Furthermore, it is to be understood that the terms “comprise,” “comprising,” and “comprises” as they relate to various aspects, elements, and features of the disclosed invention also include the more limited aspects of “consisting essentially of” and “consisting of.”

Unless defined otherwise, all technical and scientific terms used herein have the same meaning as commonly understood by one of ordinary skill in the art to which this invention belongs. In this specification and in the claims which follow, reference will be made to a number of terms that shall be defined herein.

For the terms “for example” and “such as” and grammatical equivalents thereof, the phrase “and without limitation” is understood to follow unless explicitly stated otherwise. It is further understood that these phrases are used for explanatory purposes only. It is further understood that the term “exemplary,” as used herein, means “an example of” and is not intended to convey an indication of a preferred or ideal aspect.

The expressions “ambient temperature” and “room temperature” as used herein are understood in the art and refer generally to a temperature from 20° C. to 35° C.

All disclosed values also include values that fall within a ±10% variation from the disclosed value unless otherwise indicated or inferred. In other words, if a range of 1 to 10 is disclosed, then a range of about 1 to about 10 is disclosed. In such aspects, it is understood that the amount or value in question can be the exact value or a value that provides equivalent results or effects as recited in the claims or taught herein. That is, amounts, sizes, formulations, parameters, and other quantities and characteristics include both exact values but also approximate, larger or smaller values as desired, reflecting tolerances, conversion factors, rounding, measurement error, and the like, and other factors known to those of skill in the art such that equivalent results or effects are obtained. In some circumstances, the value that provides equivalent results or effects cannot be reasonably determined. In general, an amount, size, formulation, parameter, or other quantity or characteristic is “about,” “approximate,” or “at or about,” whether or not expressly stated to be such. Where “about,” “approximate,” or “at or about” is used before a quantitative value, the parameter also includes the specific quantitative value itself unless expressly stated otherwise.

As used herein, the term or phrase “effective,” “effective amount,” or “conditions effective to” refers to such amount or condition that is capable of performing the function or property for which an effective amount or condition is expressed. As will be pointed out below, the exact amount or particular condition required will vary from one aspect to another, depending on recognized variables such as the materials employed and the processing conditions observed. Thus, it is not always possible to specify an exact “effective amount” or “condition effective to. ” However, it should be understood that an appropriate, effective amount will be readily determined by one of ordinary skill in the art.

When a range is expressed, a further aspect includes from the one particular value and to the other particular value. For example, where the stated range includes one or both of the limits, ranges excluding either or both of those included limits are also included in the disclosure, e.g., the phrase “x to y” includes the range from ‘x’ to ‘y’ as well as the range greater than ‘x’ and less than ‘y’. The range can also be expressed as an upper limit, e.g., ‘x, y, z, or less’ and should be interpreted to include the specific ranges of ‘x,’ ‘y,’ ‘z,’ ‘about x,’ ‘about y,’ and ‘about z’ as well as the ranges of ‘less than x,’ ‘less than y, or ‘less than z,’ or ‘less than about x,’ ‘less than about y, and ‘less than about z.’ Likewise, the phrase ‘x, y, z, or greater’ should be interpreted to include the specific ranges of ‘x,’ ‘y,’ ‘z,’ ‘about x,’ ‘about y,’ and ‘about z’ as well as the ranges of ‘greater than x,’ greater than y,’ ‘greater than z,’ or ‘greater than about x,’ greater than about y,’ ‘greater than about z.’ In addition, the phrase “‘x’ to ‘y’,” where ‘x’ and ‘y’ are numerical values, also includes “about ‘x’ to about ‘y’.”

Such a range format is used for convenience and brevity and, thus, should be interpreted flexibly to include not only the numerical values explicitly recited as the limits of the range but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5% but also include individual values (e.g., 1%, 2%, 3%, and 4%) and the sub-ranges (e.g., 0.5% to 1.1%; 5% to 2.4%; 0.5% to 3.2%, and 0.5% to 4.4%, and other possible sub-ranges) within the indicated range.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6 and any whole and partial increments therebetween. This applies regardless of the breadth of the range.

In still further aspects, when the specific values are disclosed between two end values, it is understood that these end values can also be included.

In still further aspects, when the range is given, and exemplary values are provided, it is understood that any ranges can be formed between any exemplary values within the broadest range. For example, if individual numbers 1, 2, 3, 4, 5, 6, 7, etc. are disclosed, then the ranges 1-7, 2-7, 3-7, 4-7, 5-7, 6-7, 1-6, 1-5, 1-4, 1-3, 1-2, 2-6, 2-5, etc. are also disclosed.

References in the specification and concluding claims to parts by weight of a particular element or component in a composition denote the weight relationship between the element or component and any other elements or components in the composition or article for which a part by weight is expressed. Thus, in a mixture containing 2 parts by weight of component X and 5 parts by weight, components Y, X, and Y are present at a weight ratio of 2:5 and are present in such a ratio regardless of whether additional components are contained in the mixture.

A weight percent (wt. %) of a component, unless specifically stated to the contrary, is based on the total weight of the formulation or composition in which the component is included.

It will be understood that when an element is referred to as being “connected” or “coupled” to another element, it can be directly connected or coupled to the other element, or intervening elements may be present. In contrast, when an element is referred to as being “directly connected” or “directly coupled” to another element, there are no intervening elements present. Other words used to describe the relationship between elements or layers should be interpreted in a like fashion (e.g., “between” versus “directly between,” “adjacent” versus “directly adjacent,”“on”versus “directly on”).

As used herein, the term “and/or” includes any and all combinations of one or more of the associated listed items.

It will be understood that the terms “first,” “second,” etc., may be used herein to describe various elements, components, regions, layers, and/or sections. These elements, components, regions, layers, and/or sections should not be limited by these terms. These terms are only used to distinguish one element, component, region, layer, or section from another element, component, region, layer, or section. Thus, a first element, component, region, layer, or section discussed below could be termed a second element, component, region, layer, or section without departing from the teachings of example aspects.

As used herein, the term “substantially” means that the subsequently described event or circumstance completely occurs or that the subsequently described event or circumstance generally, typically, or approximately occurs.

Still further, the term “substantially” can, in some aspects, refer to at least 90%, at least 95%, at least 99%, or 100% of the stated property, component, composition, or other condition for which substantially is used to characterize or otherwise quantify an amount.

In other aspects, as used herein, the term “substantially free,” when used in the context of a composition or component of a composition that is substantially absent, is intended to refer to an amount that is then 1% by weight, e.g., less than 0.5% by weight, less than 0.1% by weight, less than 0.05% by weight, or less than 0.01% by weight of the stated material, based on the total weight of the composition.

Spatially relative terms, such as “beneath,” “below,” “lower,” “above,” “upper,” “bottom,” “top,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the term “below” can encompass both an orientation of above and below. The device may be otherwise oriented (rotated 90 degrees or at other orientations), and the spatially relative descriptors used herein are interpreted accordingly.

Terms such as “proximal,” “distal,” “radially outward,” “radially inward,” “outer,” “inner,” and “side” describe the orientation and/or location of portions of the components or elements within a consistent but arbitrary frame of reference which is made clear by reference to the text and the associated drawings describing the components or elements under discussion. Such terminology can include the words specifically mentioned above, derivatives thereof, and words of similar import.

As used herein, “treating” and “treatment” generally refer to obtaining a desired pharmacological or physiological effect. The effect can be but does not necessarily have to be prophylactic in preventing or partially preventing a disease, symptom, or condition. The effect can be therapeutic regarding a partial or complete cure of a disease, condition, symptom, or adverse effect attributed to the disease, disorder, or condition. The term “treatment” as used herein can include any treatment of a disorder in a subject, particularly a human. It can include any one or more of the following: (a) preventing the disease from occurring in a subject who may be predisposed to the disease but has not yet been diagnosed as having it; (b) inhibiting the disease, i.e., arresting its development; and (c) relieving the disease, i.e., mitigating or ameliorating the disease or its symptoms or conditions. The term “treatment,” as used herein, can refer to both therapeutic treatment alone, prophylactic treatment alone, or both therapeutic and prophylactic treatment. Those in need of treatment (i.e., subjects in need thereof) can include those already with the disorder or those in which the disorder is to be prevented. As used herein, the term “treating” encompasses both inhibiting the disease, disorder, or condition, e.g., impeding its progression, and relieving the disease, disorder, or condition, e.g., causing regression of the disease, disorder, or condition. Treating the disease, disorder, or condition can include ameliorating at least one symptom of the particular disease, disorder, or condition, even if the underlying pathophysiology is not affected, e.g., such as treating the pain of a subject by administration of an analgesic agent even though such agent does not treat the cause of the pain.

Some aspects described herein relate to methods. It should be understood that such methods can be implemented using computer. That is, where the method or other events are described herein, it should be understood that they may be performed by a computing device having a processor and a memory. Memory of a computing device is also referred to as a non-transitory computer-readable medium, which can include instructions or computer code for performing various computer-implemented operations. The computer-readable medium (or processor-readable medium) is non-transitory in the sense that it does not include transitory propagating signals per se (e.g., a propagating electromagnetic wave carrying information on a transmission medium such as space or a cable). The media and computer code (also referred to as code) may be those designed and constructed for a specific purpose or purpose. Examples of non-transitory computer-readable media include, but are not limited to magnetic storage media such as hard disks, floppy disks, and magnetic tape; optical storage media such as Compact Disc/Digital Video Discs (CD/DVDs), Compact Disc-Read Only Memories (CD-ROMs), and holographic devices; magneto-optical storage media such as optical disks; carrier wave signal processing modules, Read-Only Memory (ROM), Random-Access Memory (RAM) and/or the like. One or more processors can be communicatively coupled to the memory and operable to execute the code stored on the non-transitory processor-readable medium. Examples of processors include general purpose processors (e.g., CPUs), Graphical Processing Units, Field Programmable Gate Arrays (FPGAs), Application Specific Integrated Circuits (ASICs), Digital Signal Processor (DSPs), Programmable Logic Devices (PLDs), and the like. Examples of computer code include, but are not limited to, micro-code or micro-instructions, machine instructions, such as those produced by a compiler, code used to produce a web service, and files containing higher-level instructions that are executed by a computer using an interpreter. For example, aspects may be implemented using imperative programming languages (e.g., C, Fortran, etc.), functional programming languages (Haskell, Erlang, etc.), logical programming languages (e.g., Prolog), object-oriented programming languages (e.g., Java, C++, etc.), or other suitable programming languages and/or development tools. Additional examples of computer code include but are not limited to, control signals, encrypted code, and compressed code.

While aspects of the present invention can be described and claimed in a particular statutory class, such as the system statutory class, this is for convenience only, and one of ordinary skill in the art will understand that each aspect of the present invention can be described and claimed in any statutory class. Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred in any respect. This holds for any possible non-express basis for interpretation, including matters of logic with respect to the arrangement of steps or operational flow, plain meaning derived from grammatical organization or punctuation, or the number or type of aspects described in the specification.

The present invention may be understood more readily by reference to the following detailed description of various aspects of the invention and the examples included therein and to the Figures and their previous and following description.

### Single-Use Container and Apparatus

In certain aspects, disclosed herein is a single-use container for forming a therapeutic amount of nitric oxide. Such a container is configured to be positioned within an apparatus that is configured to deliver the therapeutic amount of nitric oxide to a mammal. Any of the known in-the-art apparatuses that are compatible with the disclosed herein single-use container can be utilized. Some exemplary apparatuses are discussed below in more detail. It is understood that the term “mammal” refers to any mammal that requires a therapeutic amount of nitric oxide for whatever reason. In certain exemplary and unlimiting aspects, the mammal is a human patient. However, it is understood that the formed nitric oxide can be delivered to any other known mammal if needed.

It is understood that the single-use container disclosed herein can be used for medical purposes and, more specifically, for forming a desired amount of nitric oxide that can then be delivered to the subject. In aspects disclosed herein, the single-unit use container is utilized upon request and, by the end of the use, can be discarded and/or recycled if needed.

In still further aspects, the single-use container disclosed herein can comprise a housing defined by a proximal edge and a distal edge. In still further aspects, the housing can comprise a first chamber and a second chamber. In certain aspects, the first chamber comprises N₂O₄. In other aspects, the second chamber comprises an antioxidant material. In still further aspects, the first chamber is sealed. It is understood that the term “sealed” as used herein refers to the first chamber that can be fully sealed, or it can be partially sealed, or at least partially sealed. In still further aspects, the first chamber and the second chamber are positioned relative to each other such that when the single-use container is inserted into the apparatus and is activated, the first chamber can be unsealed to allow the N₂O₄ to be in fluid communication with the antioxidant material in the second chamber to produce nitric oxide. It is further understood that the unsealing process does not have to fully unseal the first chamber. In such aspects, the first chamber can be partially unsealed.

In certain aspects, the N₂O₄ is present as a liquid. In yet still further aspects, the liquid N₂O₄ can be in equilibrium with NO₂. Yet in still further aspects, the liquid N₂O₄ is in equilibrium with gaseous nitrogen dioxide (NO₂) or a gaseous mixture of dinitrogen tetroxide N₂O₄ and nitrogen dioxide (NO₂). In yet still further aspects, the first chamber can also comprise an amount of NO₂ or an amount of the gaseous mixture of dinitrogen tetroxide N₂O₄ and nitrogen dioxide (NO₂). In certain aspects, the first chamber can comprise a liquid phase and a gas space.

In such exemplary and unlimiting aspects, the amount of NO₂ or an amount of the gaseous mixture of dinitrogen tetroxide N₂O₄ and nitrogen dioxide (NO₂) can be found in the gas space.

In still further aspects, it is understood that N₂O₄ can be present not as a pure liquid form. For example, N₂O₄ can be present as incorporated in additional media. For example, and without limitations, N₂O₄ can be present as a gel or any other matrix. It is understood that in such aspects, the media and/or matrix are not reactive towards N₂O_{4.} In such aspects, the media or matrix serves as a host of N₂O₄ to improve, for example, and without limitations, the safety and accessibility of the device. In such aspects, the matrix containing N₂O₄ can be positioned within the first chamber, and NO₂ can be formed during the activation of the first chamber. In still further aspects, the NO₂ formed during the activation of the first chamber can be positioned in the gas space of the first chamber.

In certain aspects, the antioxidant can be disposed within a solid matrix. In some aspects, the solid matrix can be a porous matrix. Yet, in other aspects, the solid matrix can take any form that allows for a high surface area and penetration of the antioxidant within the solid matrix. In still further aspects, it is understood, without being bound by any theory, that the high surface area of the solid matrix allows for a more efficient interaction between the antioxidant and N₂O₄ and/or NO₂, producing nitric oxide. In still further aspects, the antioxidant can be present without the solid matrix. For example, and without limitations, the antioxidant can be presented as a fluid. In certain aspects, the fluid can be stationary. Yet, in other aspects, the fluid can be continuously flowed through the second chamber to ensure a desired mixing between the antioxidant and the N₂O₄ and/or NO₂, producing nitric oxide. In further aspects, the second chamber can have agitation elements that allow for the desired mixing.

Antioxidants can comprise any known in the art antioxidants. In some aspects, the antioxidant can comprise ascorbic acid; alpha-, beta-, gamma-, or delta tocopherol; alpha-, beta-, gamma-, or delta-tocotrienol; polyphenols; beta-carotene; or a combination thereof. In still further aspects, the media comprising antioxidants can also comprise at least some amount of moisture.

In aspects where the solid matrix is present, such a solid matrix can comprise silica gel or other suitable high-surface area wettable material that is wetted, coated, or impregnated with the antioxidant. Nitrogen dioxide can react with an aqueous solution of the antioxidant to produce nitric oxide, following the following reactions:

a. 6NO₂(gas)+3H₂O (liq.)→3HNO₃ (liq.)+3HNO₂ (liq.) Eq. 1a

b. 3HNO₂(liq.)→HNO₃ (liq.)+2NO (gas)+H2O (liq.) Eq. 1b

Yet, in other aspects, the NO can be formed according to Eq. 2a-2b:

C₆H₈O₆+NO₂=>C₆H₆O₆+NO 30 H₂O Eq. 2a

3NO₂+H₂O=>2HNO₃+NO Eq. 2b

Ascorbic acid-wetted solid matrices (or other suitable antioxidant-containing materials) can be functionally similar to the media described in disclosed in U.S. Pat. Nos. 8,607,785, 8,944,049, 9,604,028, 10,926,054, 11,744,978, the contents of which are incorporated herein in their whole entirety.

Any of the antioxidants disclosed above can be present in an aqueous solution at concentrations ranging from 0 to 50 wt %, including exemplary values of 1 wt %, 5 wt %, 10 wt %, 15 wt %, 20 wt %, 25 wt %, 30 wt %, 35 wt %, 40 wt %, and 45 wt %. It is understood that antioxidants can be present in any amount that falls between any two disclosed above values, or they can be present in any range that can be formed by any two values that fall within the broadest range. For example, the antioxidant can be present in an amount of 1 wt % to 40 wt %, 1 wt % to 32 wt %, 5 wt % to 32 wt %, 15 wt % to 45 wt %, 1 wt % to 15 wt %, 10 wt % to 15 wt %, and so on. It may be desirable for the antioxidant concentration to be at or below the room temperature solubility limit because, at higher concentrations, the antioxidant may precipitate out, which could produce inconsistent nitric oxide conversion dynamics. It should be understood, however, that it may be possible to further increase the concentration of antioxidants by heating an antioxidant solution before wetting the solid matrix.

In still further aspects, the antioxidant is dispersed within a media comprising a support material having a specific surface area of 350 to 5000 m²/g, including exemplary values of 400 m²/g, 500 m²/g, 600 m²/g, 700 m²/g, 800 m²/g, 900 m²/g, 1000 m²/g, 1250 m²/g, 1500 m²/g, 1750 m²/g, 2000 m²/g, 2250 m²/g, 2500 m²/g, 2750 m²/g, 3000 m²/g, 3250 m²/g, 3500 m²/g, 3750 m²/g, 4000 m²/g, 4250 m²/g, 4500 m²/g, 4750 m²/g, and 4990 m²/g. It is understood that the specific surface area can be in any range formed between any two foregoing values. For example, and without limitations, the specific surface area can be 350 to 4500 m²/g, 400 to 5000 m²/g, 400 to 4000 m²/g, 500 to 5000 m²/g, 500 to 4000 m²/g, 500 to 3000 m²/g, or 500 to 2000 m²/g, and so on.

Yet in other aspects, the second chamber can comprise media that is described in U.S. Patent Application No. 63/573,175, the content of which is incorporated herein is all entirety.

In still further aspects, the second chamber can be filled with any of the disclosed herein or in U.S. Patent Application No. 63/573,175 antioxidant-containing media prior to sealing.

In yet still further aspects, the antioxidant-containing media can be compressed during assembly to form a precise packing, and this compression can be applied by the process of screwing the second chamber onto a flow-directing unit or screwing the flow-directing unit onto the second chamber as disclosed in more detail below. Yet in other aspects, the attachment of the second chamber to the flow-directing unit can be done by other than screwing methods. In certain aspects, the second chamber can be tightly inserted into the flow-directing unit and affixed to it. In yet still further aspects, the second chamber can be affixed to the first chamber and/or the flow-directing unit by any other known in the art methods to ensure, if needed, additional compression of the antioxidant media.

In still further aspects, the affixing of the second chamber allows to provide a sealed flow path between the first and second chambers. In still further aspects, the sealed flow path can be achieved by the presence of O-rings and retaining features such as a latch.

In certain aspects, the second chamber can also be sealed. In still further aspects, the second chamber can be partially sealed or at least partially sealed. In some aspects, the second chamber is unsealed when the single-use container is inserted into the apparatus. In yet other aspects, the second chamber can be partially unsealed.

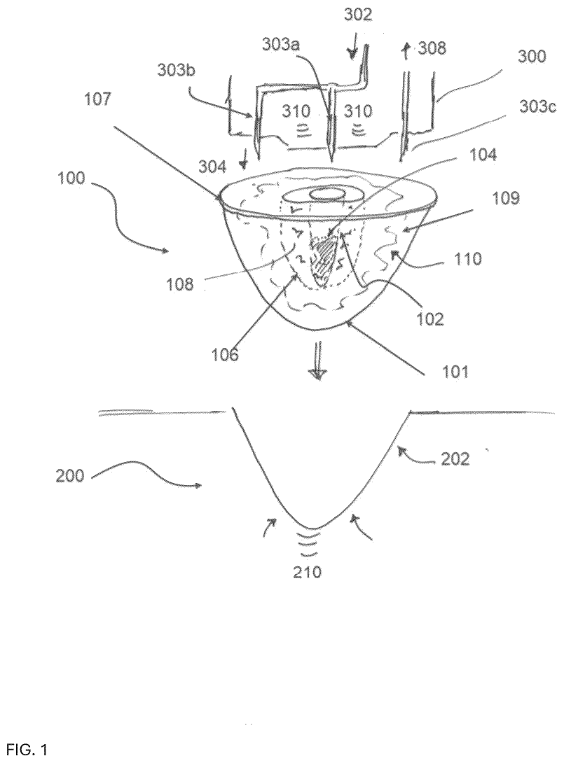

The exemplary and unlimiting single-use containers are shown in FIGS. 1-6, 8-10F, 13A-26E, 28-30D, and 32A-37B.

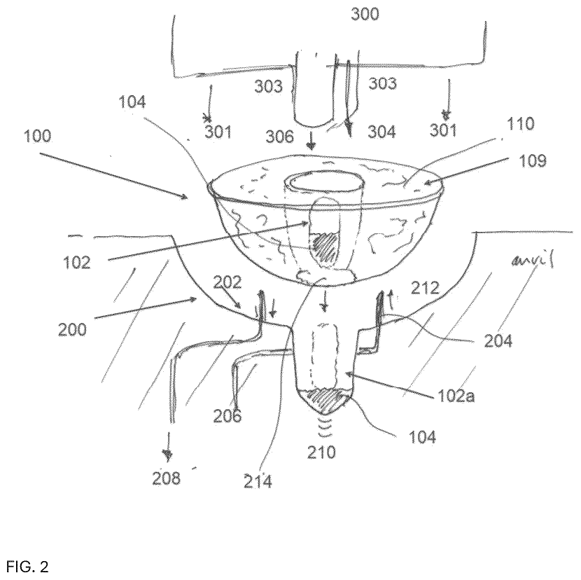

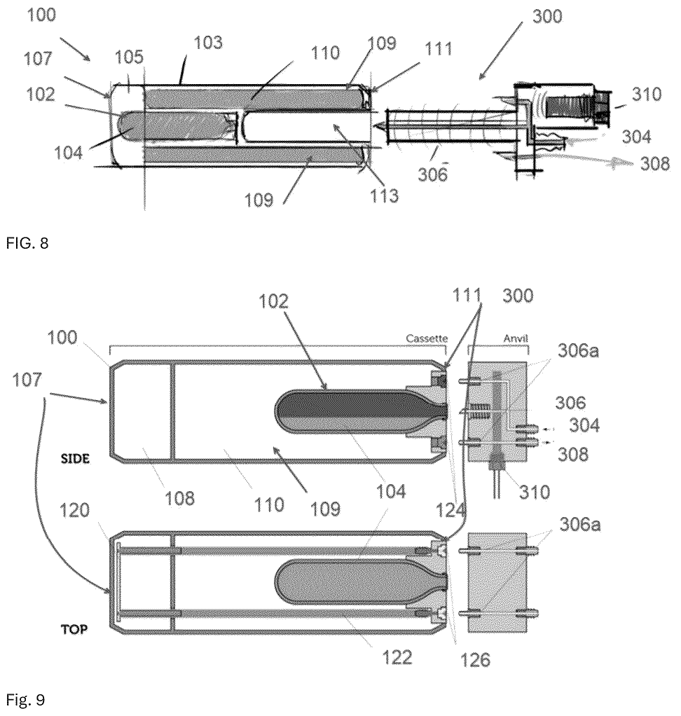

It is understood that the single-use container can have any desirable shape and form. The shape and form can be chosen to be ergonomic and to be capable of engaging with the delivery apparatus in the desired way. Referring to FIGS. 1 and 2, the single-use container 100 can have an exemplary shape of a pod. Again, it is understood that such a shape is exemplary and non-limiting. The single-use container 100 is shown in FIGS. 1 and 2 have a first chamber 102 that can comprise a liquid N₂O₄ 104 and a second chamber 109 that comprises an antioxidant 110. It can be seen that in this configuration, the first and second chambers are positioned concentrically to each other. For example, FIGS. 6 and 8 show configurations where the positioning of the first 102 and the second 109 chambers can be also assumed to be concentrical.

In still further aspects, the first chamber can be positioned within the second chamber. In such aspects, the two chambers do not have to share the same central axis. For example, the central axis of the first chamber can be offset from the central axis of the second chamber if desired.

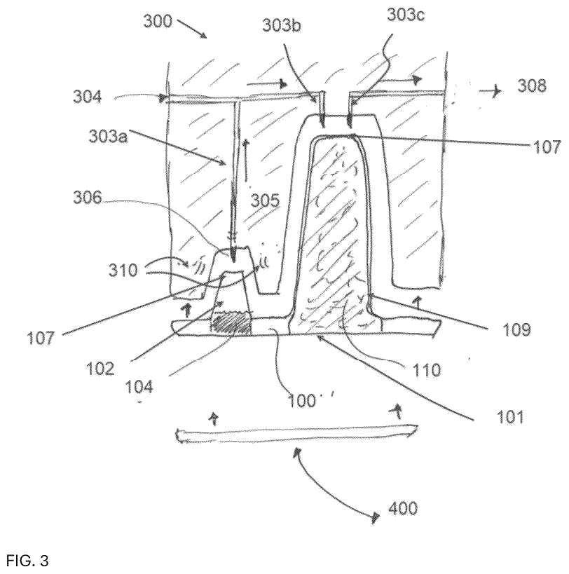

The exemplary single-use container 100, as shown in FIG. 3 has a different configuration. In this configuration, the first chamber 102 and the second chamber 109 are presented as two separated pods coupled to each other in series.

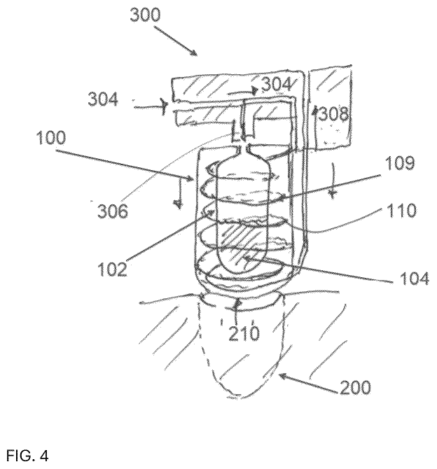

FIG. 4 shows an additional exemplary configuration of the single-use container 100. In this configuration, the first chamber 102 can be surrounded by a second chamber 109. In this exemplary aspect, the second chamber 109 can be presented as a tubing that wraps around the first chamber 102. In such an exemplary configuration, the second chamber can either have a solid matrix disposed within the tubing or can only comprise a granular, powder, gel, or fluid mixture of antioxidant 110 that is configured to come in contact with N₂O₄ and/or NO₂ to produce nitric oxide.

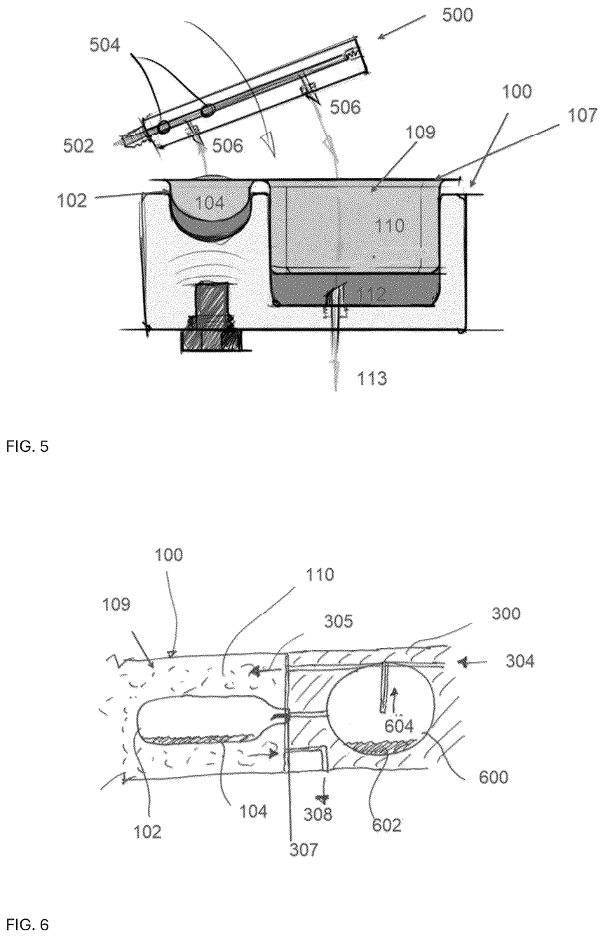

FIG. 5 shows a configuration similar to FIG. 3, where two chambers are positioned in series as two separate pods 102 and 109.

In still further aspects, and as will be shown below in more detail, when the unsealing of the first chamber occurs, the formation of the therapeutic amount of nitric oxide is initiated.

In still further aspects, the first chamber 102 is a pressure vessel. It is understood that the term “pressure vessel” as used herein refers to any vessel capable of containing (a liquid and/or gas and/or fluid and/or gel or solid) and/or withstanding pressure up to 1000 psi, up to 900 psi, up to 800 psi, up to 700 psi, up to 600 psi, or up to 500 psi. In still further aspects, the pressure vessel as described herein is capable of containing (a liquid and/or gas and/or fluid) and/or withstanding pressure of equal to or greater than 14 psi to 1000 psi, including exemplary values of 15 psi, 50 psi, 100 psi, 150 psi, 200 psi, 250 psi, 300 psi, 350 psi, 400 psi, 450 psi, 500 psi, 550 psi, 600 psi, 650 psi, 700 psi, 750 psi, 800 psi, 850 psi, 900 psi, and 950 psi. It is understood that any ranges between any two foregoing values or ranges formed by any two foregoing values can be formed. For example, and without limitations, the pressure vessel as described herein is capable of containing a liquid and/or gas and/or fluid and/or withstanding pressure of 15 psi to 600 psi, 15 psi to 500 psi, or 15 psi to 400 psi, or 100 psi to 900 psi, and so on. It is understood that the first chamber is made of a material that is capable of withstanding the disclosed above pressures and chemical conditions in which the chamber operates. In certain exemplary and limiting aspects, the first chamber can be made of stainless steel, aluminum, any conductive and inductive metals and alloys thereof, thermally conductive polymers, polymers, or any combination thereof. It is understood, however, that the materials used to form the first chamber are chosen to withstand the pressure and chemical conditions required for the first chamber operation. In certain aspects, the first chamber can also comprise an inductive element that can be heated if needed to initiate the desired reactions.

In still further aspects, the first chamber is directly filled with the N₂O₄ in a liquid form or with the N₂O₄ incorporated within a media. Yet in still further aspects, the first chamber can comprise an ampule filled with the N₂O₄. In certain and unlimiting aspects, the ampule can be positioned such that a proximal edge of the ampule is aligned with a proximal edge of the first chamber, and wherein the ampule is stationary within the first chamber.

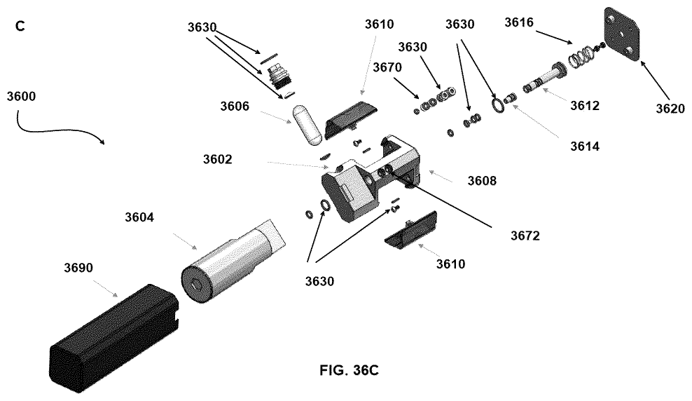

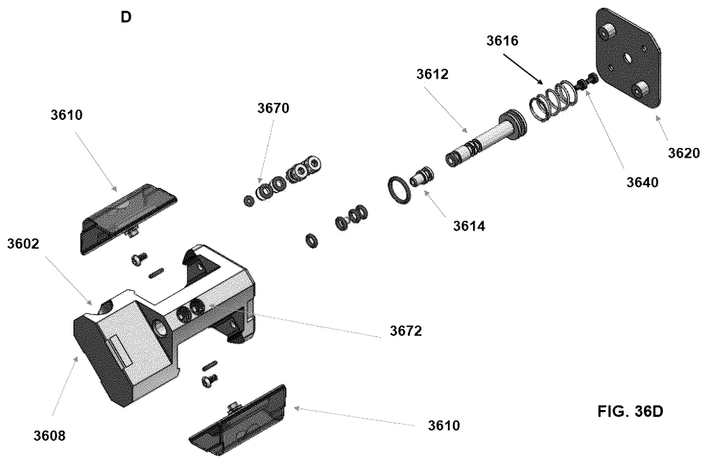

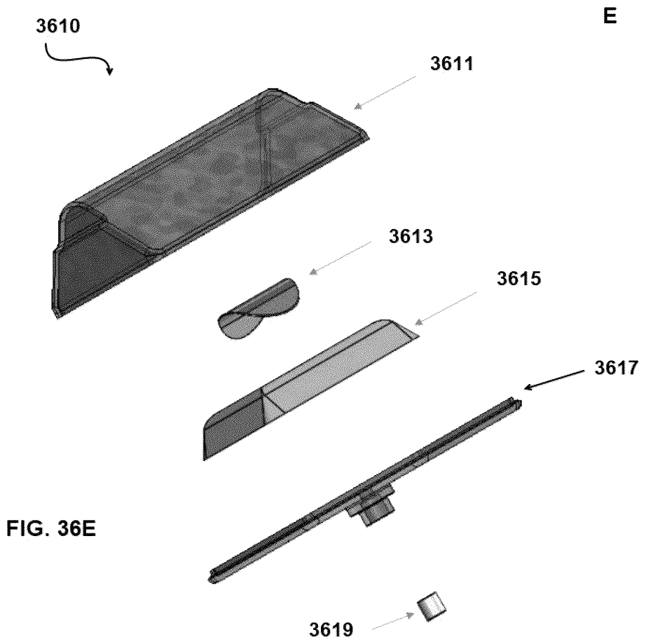

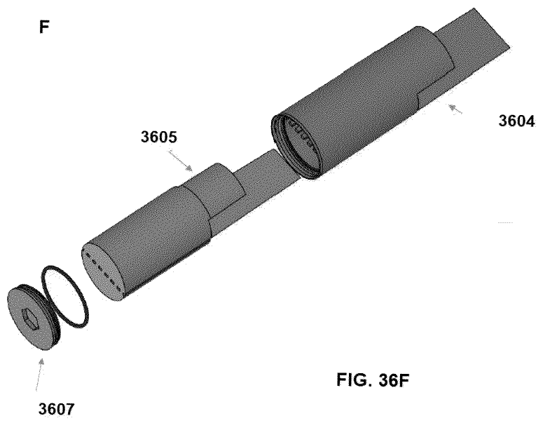

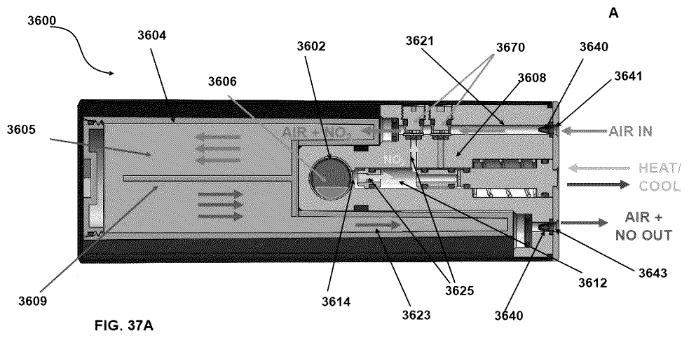

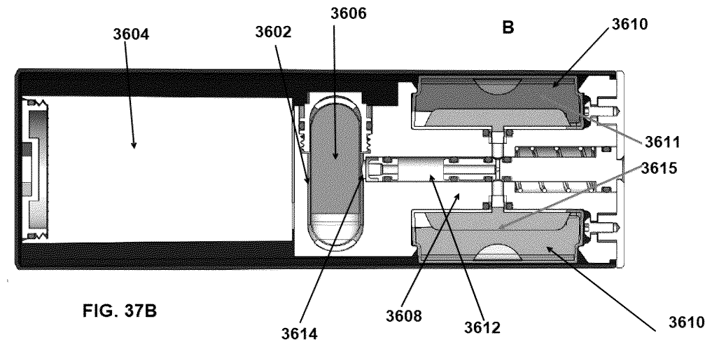

Yet in other exemplary and unlimiting aspects, the ampule can have any other positioning within the first chamber that suits the desired application. For example, as shown in FIGS. 36B-37B, and as discussed in more detail below, the ampule can be such that a middle portion of ampule 3606 is in parallel with a proximal edge (as it can be defined by a base plate 3620 in FIG. 36C), of the single-use container 3600, in other words, the ampule is positioned perpendicular to a main longitudinal axis L (FIG. 36A) of the single-use container.

In such aspects, the ampule can be a glass ampule, for example. Yet, in other aspects, the ampule can be positioned without the presence of the first chamber. Yet in still further aspects, the first chamber can be a receptacle or a mounting space that is configured to receive the ampule (FIGS. 36B-36F). In certain aspects, the ampule can be the first chamber. In such exemplary aspects, the breaking or piercing of the ampule results in directing its contents, e.g., the N₂O₄/NO₂, to a controlled/sealed path.

In still further aspects, and as shown herein, the housing of the single-use container can also comprise one or more inerting chambers. In such aspects, the one or more inerting chambers can be defined by a volume within the single-use container, or it can be a separate container that is coupled with other portions of the single-use container.

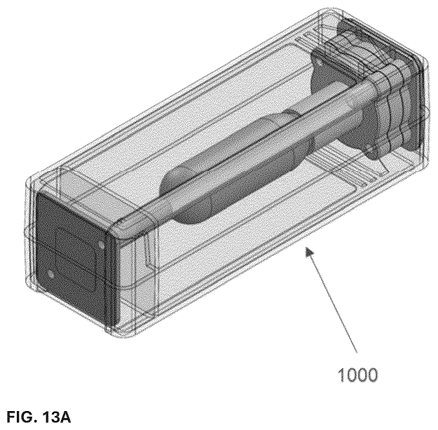

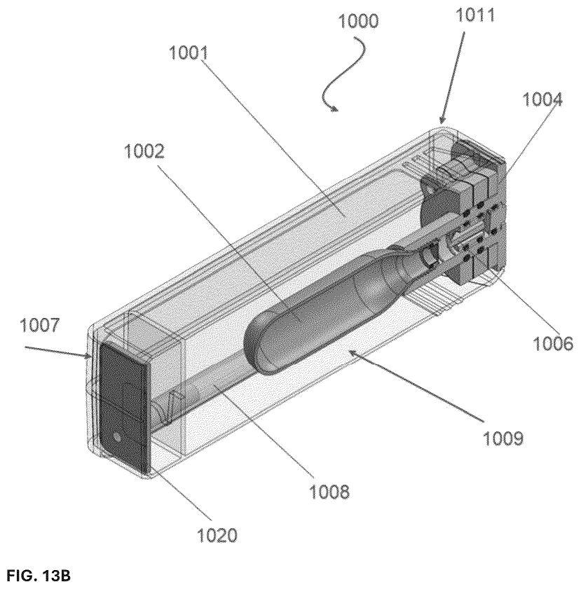

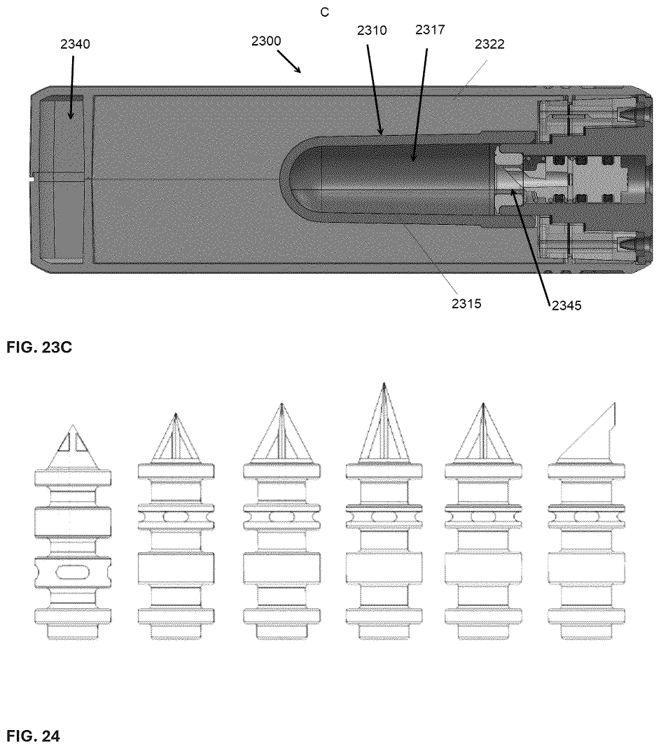

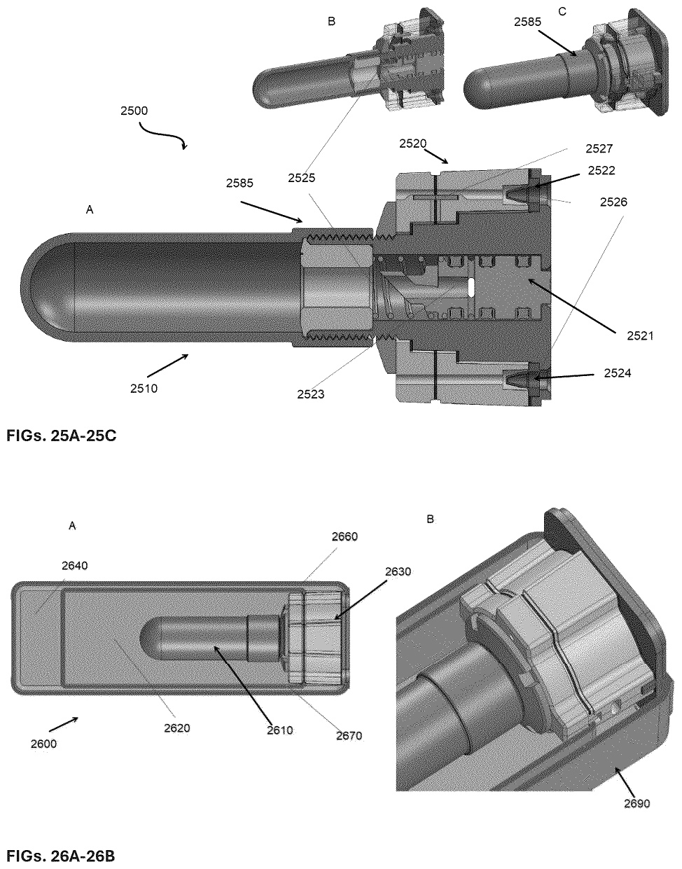

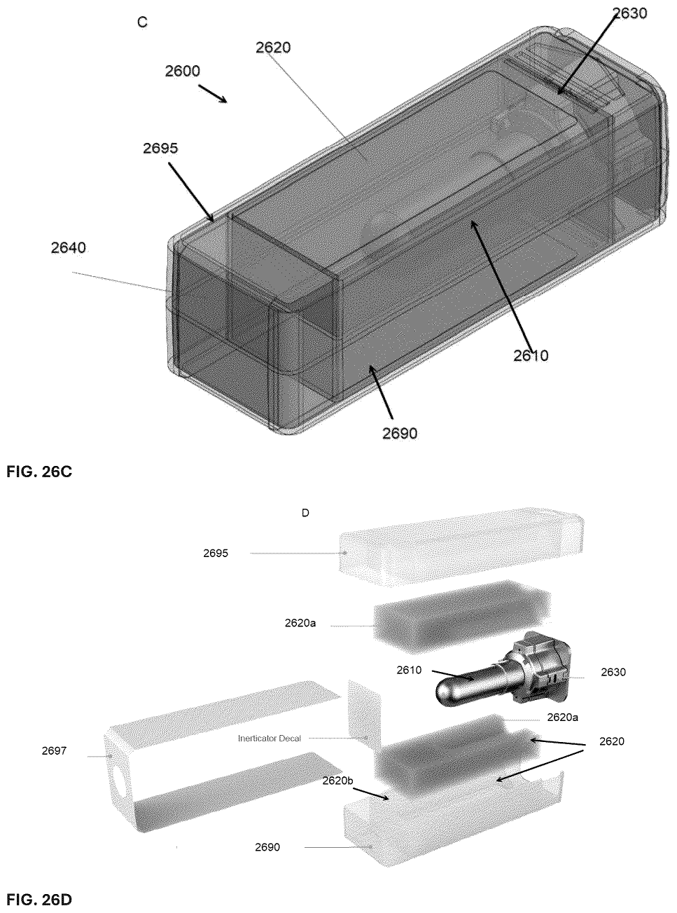

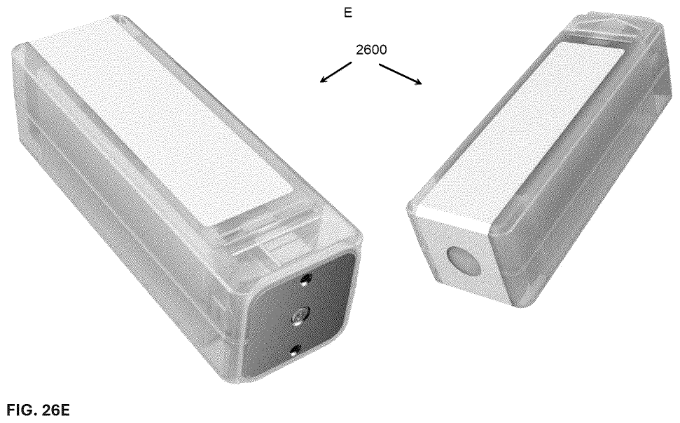

For example, the inerting chamber 106, as shown in FIG. 1, can also comprise an inerting material 108 that is configured to inert liquid N₂O₄ or gaseous NO₂ if exposed. In such exemplary and unlimiting aspects, the inerting chamber (it is understood that these two terms can be used interchangeably) can be positioned such that any inadvertent leak of N₂O₄ or NO₂ can be captured and contained within the inerting material. Yet, in other aspects, the inerting chamber can be utilized when the single-use container is consumed. In such aspects, any remaining N₂O₄ or NO₂ at the end of the use of the single-use container can be conveyed to and contained within the inerting chamber. The inerting chamber can have different positioning or configurations. For example, the inerting chamber 106 shown in FIG. 1 is concentric to the first chamber 102. While the inerting chamber 1020, as shown in FIG. 13B can be positioned in a distal portion of the single-use container. Similarly, the inerting chambers 2140 (FIGS. 21A), 2340 (FIGS. 23A), 2640 (FIG. 26A) are positioned in the distal portion of the single-use container.

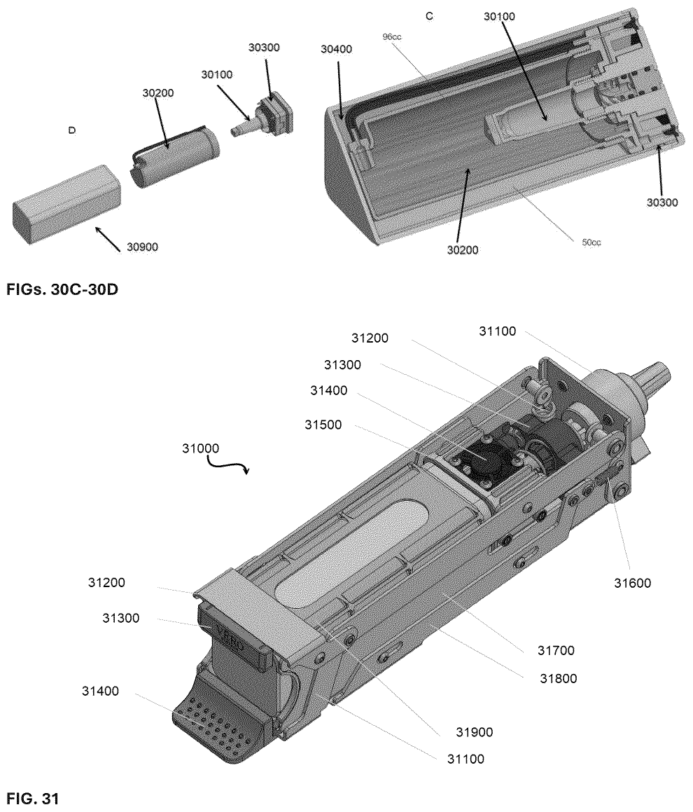

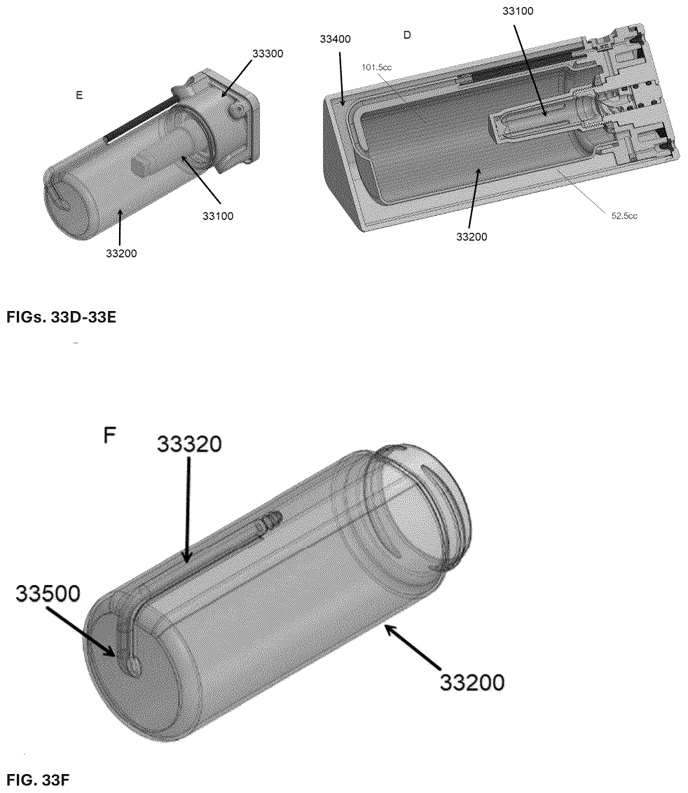

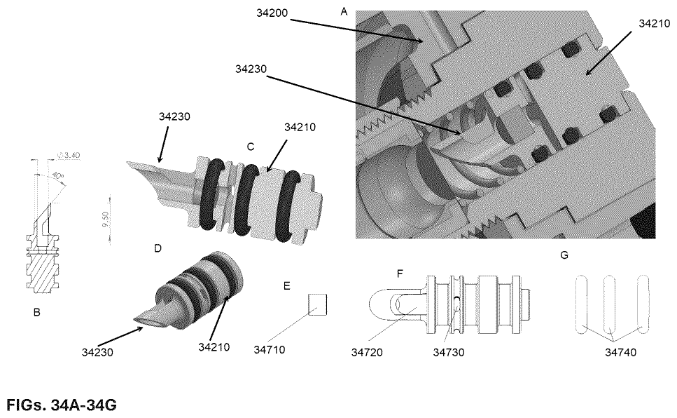

In yet other aspects, the inerting chamber can at least partially encompass the second chamber (3040 (FIGS. 30A), 3340 (FIGS. 33A, 33D).

In certain aspects, the one or more inerting chambers can be in fluid communication with the first chamber. Yet in still further aspects, the single-use container can comprise two inerting chambers 3610 (FIGS. 36B-36F) that are positioned in the proximal portion of the single-use container 3600. The exploded view of one of the two inerting chambers 3610 is shown in FIG. 36E. The inerting chamber can be defined, for example, and without limitations, by the housing comprising a plate 3617 that attaches the chamber to the proximal portion of the single-use container, the top cover (or housing) 3611 that hosts the inerting material 3615. In certain aspects, the inerting chamber can comprise a gas indicator 3613, for example, NO₂ gas. In still further aspects, the inerting chamber can comprise a filter 3619.

In certain aspects, the one or more inerting chambers can take the form of an open volume filled with the inerting material. In such exemplary and unlimiting aspects, the liquid N₂O₄ and NO₂ gas can be distributed throughout this inerting volume once directed by an appropriate mechanism. For example, and as disclosed in detail below, such an appropriate mechanism can be a flow-directing unit.

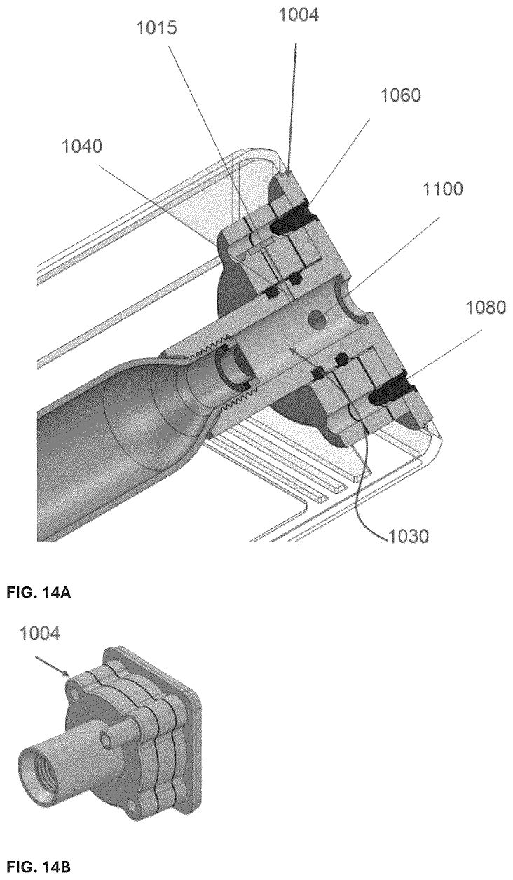

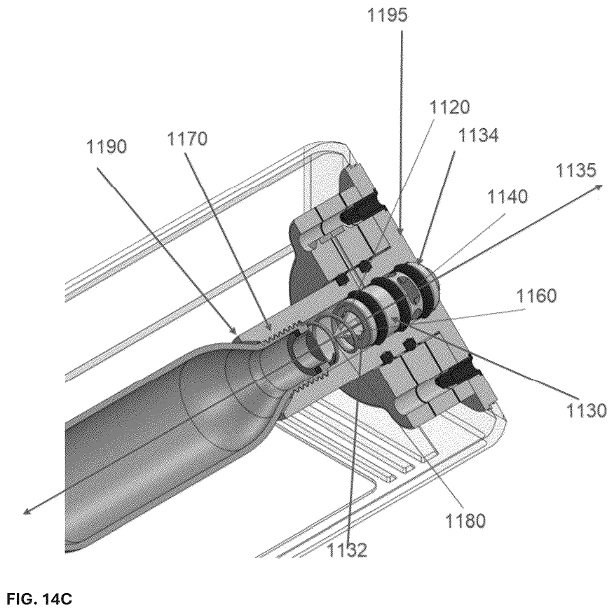

In certain exemplary and unlimiting aspects, the flow-directing unit is comprised of several components. In certain exemplary and unlimiting aspects, and as shown for example in FIGS. 14-19, the flow-directing unit comprises a base plate or first stage. In further aspects, the flow-directing unit can comprise a bed where a valve resides. In certain aspects, the valve can have a needle end and one or more side ports, inlet and outlet orifices with optional valves that are closed (or sealed), but openable (or unsealed) by mating surfaces in an anvil upon insertion of the single-use container into the mechanism and contact with the anvil.

In further aspects, the flow-directing unit can further comprise a second stage, which houses an orifice plate assembly that allows installation and removal of orifice plates with different size holes and also contains the flow paths that enable fluid communication between the first chamber and second chamber as well as the first chamber and the one or more inerting chambers depending upon the position of the spool valve. In still further exemplary and unlimiting aspects, the second stage connects with the first chamber through a threaded connection and mates to the second chamber through a bayonet-style threaded feature. In yet still further aspects, the second stage can also contain a flow path that connects the air/gas path from the first stage to the second chamber, as well as a flow path from the second chamber through the first stage to exit the single-use container. In still further aspects, the second stage can contain a frit/filter receptacle and frit/filter to prevent debris or particulates from exiting the second chamber.

In still further aspects, the valve is spring loaded to seal closed in a shipping or inerting position such that no gases leak from the single-use container to the ambient atmosphere. In still further aspects, the valve can contain a needle, for example, a beveled needle, comprising a side port that can pierce a metal burst disc (or a foil) as well as crack an ampule when advanced by the firing pin. In still further aspects, the valve can contain a frit or filter receptacle and frit/filter in its internal lumen to prevent debris or particulates from exiting the first chamber. Yet in other aspects, the distal end of the valve can comprise unsealing means that are different from the needle. In such aspects, the unsealing means 3614 (FIGS. 36B-36F) are configured to break ampule 3606. In this configuration, since the ampule is positioned perpendicularly to the valve, the breakage point can be somewhere in the middle of the ampule. The unsealing means 3614 does not have to have a sharp edge and can break the ampule based on the applied force. In still further aspects, the unsealing means can at least partially remain within the ampule wall after unsealing, thus preventing the shards or any other debris or particles from entering the internal lumen of the valve. In still further aspects, the unsealing means can also comprise a filter or a frit that can further prevent undesirable migration of the debris.

In still further aspects, the flow-directing unit can also contain a burst disc, which is captured and sealed upon the mating of the first chamber with the first stage.

The first chamber can also accommodate an ampule securing device, which holds an ampule of N₂O₄ in a fixed position within the first chamber to enable accurate breakage when contacted by the needle end of the valve and also secures the ampule during shipping, so it is not rattling around and is protected from accidental breakage.

A more detailed description is disclosed below and in the enclosed figures.

In other exemplary and unlimiting aspects, the inerting chamber can take the form of a tubular volume filled with the inerting materials. In other aspects, and as shown in FIGS. 36B-37B, the single-use container can comprise two inerting chambers that are positioned around the flow-directing unit in the proximal portion of the single-use container. It is understood that the liquid N₂O₄ and NO₂ gases can be directed to any of the disclosed herein inerting chambers through a flow path by the appropriate mechanism, such as the disclosed below flow-directing unit.

It is understood that the inerting material is capable of neutralizing or scrubbing any N₂O₄ or NO₂ that has not reacted or leaked. The inerting material can comprise any material capable of reacting with N₂O₄ or NO₂. For example, and without limitations, the inerting material can comprise a soda lime. In other exemplary and unlimiting aspects, the inerting material can comprise a Sodasorb, which is a mixture of sodium hydroxide and calcium oxide.

In certain aspects, the inerting material can comprise an indicator or a sensor showing the user that the inerting material was used. Such exemplary and unlimiting indicator 3613 is shown in FIG. 36E. For example, and without limitations, the inerting material can comprise a colorimetric indicator or sensor that is configured to change color when the inerting material interacts with N₂O₄ or NO₂. In certain aspects, the change in color can be observed by the operator. Yet, in still further aspects, the change in color can be determined by a detecting unit of the apparatus. In yet other aspects, the inerting can be detected chemically or electrically by the sensor. The detecting unit of the apparatus can then communicate with the control unit of the apparatus. In certain aspects, when the control unit of the apparatus receives a signal that the inerting material has been used, the control unit can provide a signal to the operator or a patient alerting them that the cassette or single-use container is no longer usable for therapy or of the leak or other reasons that the inerting material has been activated.

In certain aspects, as described herein and shown in the drawings, the first chamber 102 and the second chamber 109 can be separated from each other by a separating element, such that when the separating element is broken upon the single-use container activation, fluid communication between the first chamber and the second chamber is established. It is understood that the separating element can be any element known in the art that can be broken by an intermediary mechanism and/or initiation mechanism. It is further understood that the separating element does not have to have direct contact with either the first chamber or the second chamber. For example, the separating element can be seals that are unsealed by unsealing elements (such as piercing elements) of the apparatus, which is discussed in detail below.

It is understood that the single-use container can have any of the configurations disclosed herein and shown in the enclosed drawings. It is further understood that the first 102 and the second 109 chambers can be positioned anywhere within the housing, depending on the desired application and the apparatus that the single-use container is interacting with. As mentioned above, the first chamber 102 and second chamber 109 can be positioned concentrically (FIG. 1, 2, 4, 6, 8-10F, 12A-19) or in series (FIGS. 3 and 5). In certain aspects, the housing can comprise a secondary chamber 102a (FIG. 2) that can be fluidically connected with the first chamber 102. In such aspects, if desired, the liquid N₂O₄ can be released into the secondary chamber 102a. In such configurations, the formed NO can be delivered by different paths than the one disclosed in FIG. 1, depending on the specific apparatus that is used.

In still further aspects, the first chamber 102 can be positioned adjacent to a distal end 107 of housing 103 of the single-use container 100, as shown in FIG. 8. The housing can, in some aspects, be transparent, yet in other aspects, it can be opaque. It is understood that the housing itself can be formed from any materials suitable for the desired application. In still further aspects, in this configuration, the housing can have a space 113 configured to receive a matching portion of the apparatus, for example, an anvil 306, as described in more detail below. Yet in other aspects, as, for example, shown in FIG. 9, the first chamber 102 can be positioned in the proximal end 111 of the single-use container 100.

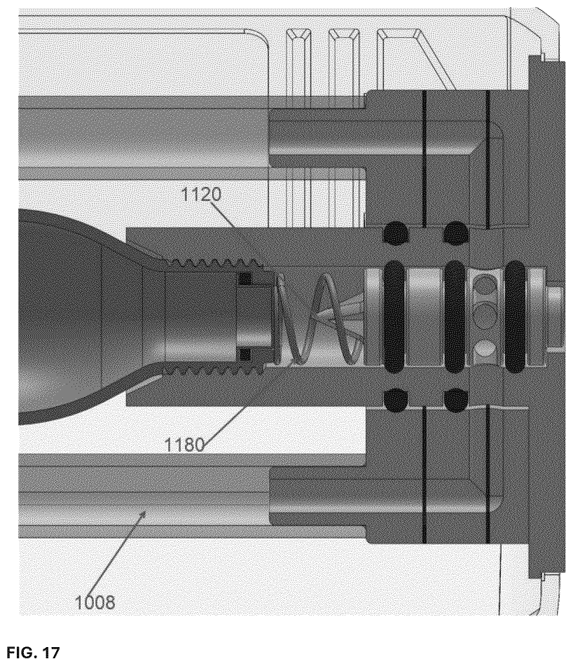

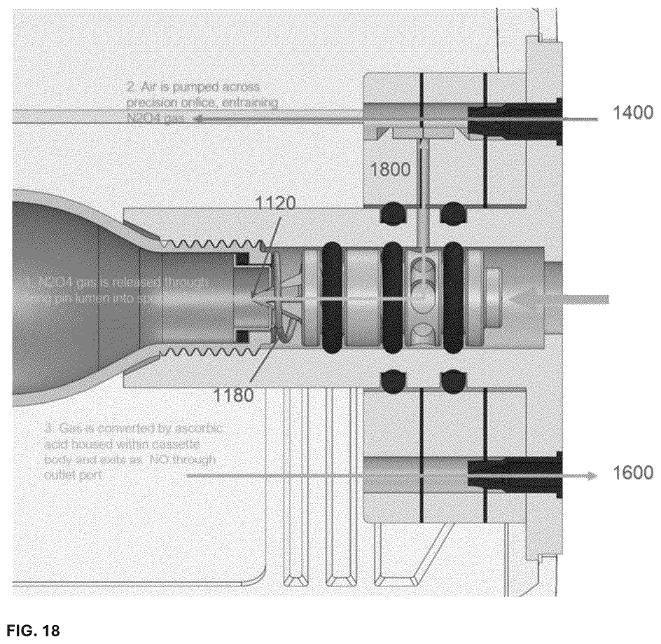

A single-use container 1000, according to an additional aspect, is shown in FIGS. 13A-16. A cross-sectional view of container 1000 is shown in FIG. 13B. The housing 1001 of container 1000 comprises a first chamber 1002 and a second chamber 1009 disposed around the first chamber. The housing can further comprise an inerting path or inerting tubing 1008 that is configured to convey and/or collect the remaining liquid N₂O₄ and/or NO₂ and to deliver it to inerting chamber 1020. Additional view of the container can be seen in FIGS. 17-19. In FIG. 18, it also shows air pumped from inlet 1400 and across a precision orifice and entering N2O4 gas, where the gas is converted by the antioxidant housed within the container, exiting as formed NO through outlet 1600.

In still further aspects, a proximal portion 1011 of the housing comprises a flow-directing unit 1004 (FIG. 13B). In such exemplary aspects, the flow-directing unit 1004 can be fluidically connected to the first chamber 1002 and the second chamber 1009 and is configured to form and/or interrupt a flow path between the first chamber 1002 and the second chamber 1009 as shown in FIG. 14A. The flow-directing unit 1004 is further fluidically connected through the orifice 1100 to the inerting chamber 1020 through the inerting tube 1008 and is configured to form and/or interrupt a flow path between the first chamber and the inerting chamber. It is understood that the inerting tubing can be an inerting flow path that is a part of the second chamber that communicates with the inerting chamber.

The flow-directing unit 1004 further comprises an inlet port 1060 that is configured to receive air from a matching portion of the apparatus, as is described in more detail below. The flow-directing unit 1004 further comprises an outlet port 1080 that is configured to match with a portion of the apparatus as it is described in more detail below and to transfer formed gas from the single-use unit to a patient. The flow-directing unit 1004 can further comprise an orifice plate 1015 having an orifice size of 1 micron to 100 microns that allows the fluid connection between the gas passage 1040 to the second chamber and the second chamber 1009. In still further aspects, the orifice plate can have an orifice size of 1 micron to 100 microns, including exemplary values of 3, 5, 10, 15, 20, 25, 30, 40, 50, 60, 70, 80, and 90 microns. It is understood that any ranges between any two foregoing values can be formed. For example, and without limitations, the orifice can have a size of 1 to 90 microns, 1 to 70 microns, 1 to 50 microns, 1 to 20 microns, 1 to 10 microns, 5 to 100 microns, 5 to 90 microns, 5 to 50 microns, 5 to 30 microns, and so on. The size of the orifice can assist in determining the flow of nitric oxide as desired. In such aspects, the flow-directing unit can be constructed such that it can accept orifice plates with different orifice sizes—this capability will create the opportunity for single-use containers that support specific dosage ranges—e.g., micro-dosing, standard, high-dose, etc.

A general view of the flow-directing unit 1004, along with a more detailed view of the unit, is shown in FIGS. 14B-14C. The flow-directing unit further comprises a valve 1130. The valve comprises a proximal end 1134 and a distal end 1132 and is configured to move along a main axis 1135 of the single-use container. It is understood that the valve is positioned within bed 1030 (FIG. 14A), configured to host the valve. In still further aspects, the valve is configured to regulate the flow path between the first chamber and the second chamber and/or the first chamber and the inerting chamber, depending on the valve position. The flow path is regulated by the positioning of the gas outlet 1140 within the valve. It is understood that any known in the art valves capable of controlling the flow as described can be utilized. In some exemplary and unlimiting aspects, the valve is a spool valve.

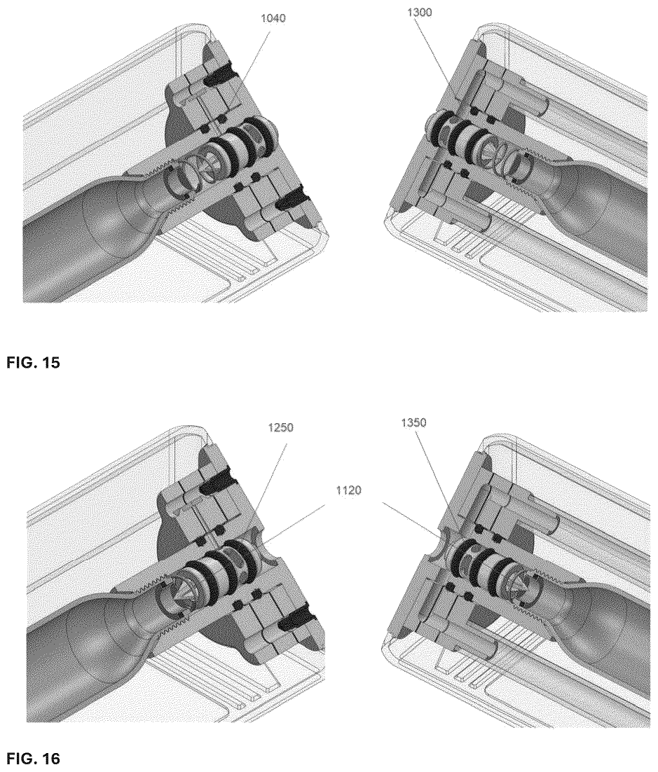

In certain aspects, before the single-use container is used, the valve is positioned such that if the first chamber is undesirably unsealed, a flow path 1300 is formed between the first chamber and the inerting chamber (FIG. 15, right side). Similarly, when the single-use container is fully utilized, the valve is positioned such that a flow path is formed between the first chamber and the inerting chamber to remove unreacted N₂O₄ and/or NO₂. It can be seen that when the single-use container is not in use, passage 1040 to the second chamber is sealed and has no flow from the valve (FIG. 15, left side).

In still further aspects, and as shown in FIG. 14C, valve 1130 comprises a needle 1120 that is positioned within the body of the valve and is configured to unseal the first chamber when activated within the apparatus. In still further aspects, the needle 1120 defines the distal end 1132 of the valve 1130. In still further aspects, the valve 1130 is a spring-loaded valve. In such aspects, the valve is coupled with spring 1180 that is configured to activate the needle 1120 to unseal the first chamber and/or also return the firing pin back at the end of the use.

In still further aspects, the valve comprises an internal lumen extending from the proximal end 1134 to the distal end 1132 of the valve. The internal lumen has a first edge and a second edge, wherein the first edge is sealed on the proximal edge of the single-use container (not shown), and the second edge 1160 defines a distal edge (or a sharp edge) of the needle firing pin 1120. It is understood that in certain aspects, the flow paths within the valve can also comprise a pledget or frit to filter the N₂O₄/NO₂ fluid that is conveyed to the micro orifice plate or capillary.

In still further aspects, a distal edge portion 1190 of the flow-directing unit comprises a mounting space 1170 configured to receive the first chamber. In certain aspects, this mounting space is configured to be heated. It is understood that any known in the art methods of heating can be utilized. In certain aspects, the mounting space can be inductively heated or heated by conduction, radiation, or by any combination thereof.

In still further aspects, and as disclosed above, the first chamber comprises a liquid N₂O₄ or N₂O₄ comprised in a solid matrix. In still further aspects, the first chamber can also comprise NO₂. When the single-use container is inserted into the apparatus and activated, the valve is moved toward the first chamber such that the needle 1120 penetrates and unseals it, allowing the release of N₂O₄/NO₂. In such aspects, the valve is positioned so that the valve's gas outlet 1250 is open towards the second chamber, creating the desired fluidic path while keeping the path 1350 to the inerting chamber closed (FIG. 16). It is also understood that the needle used in this disclosure is also configured to unseal an ampule if the ampule is used as a N₂O container.

In still further aspects, any of the disclosed herein single-use containers are configured to be received by an apparatus. The apparatus can comprise a receptacle. Such an exemplary receptacle 200 is shown in FIGS. 1-2, 4, or 400 in FIG. 3. In certain aspects, the receptacle has a mating surface 202 that is in contact with a distal end 101 of the single-use container and can also be heated by a heating element 210. Again, it is understood that any known in the art heating elements can be used.

The apparatus can further comprise an anvil. For example, FIGS. 1 and 2 show an anvil 300 that is configured to engage with at least a portion of the proximal edge 107 of the single-use container. In FIG. 3 wherein the first 102 and the second 109 chambers are disposed in series, each of the chambers has its own proximal edge 107 that can engage with the anvil 300. In such an exemplary configuration, the single-use container can be held by a plate 400, for example.

A different configuration of engagement is shown in FIG. 4. In this configuration, the second chamber 109 wraps around the first chamber 102, is positioned into a receptacle 200 of the apparatus, and is engaged with the anvil 300. In this exemplary and unlimiting aspect, the air/gas flow can enter from the left of the pod, and then it can be delivered, for example, through an orifice plate similar to the one disclosed above. Then, that flow of air/gas plus NO₂ can continue and can connect directly at the right side to the tubing that contains the antioxidant.

In the configuration shown in FIG. 5, the activation can be done by positioning anvil 500 on the proximal edge 107 of the single-use container 100 and engaging it.

In still further exemplary configurations, as shown in FIG. 6, the anvil 300 can comprise a secondary first chamber 600 that can be in fluid communication with the first chamber 102 after the single-use container 100 is inserted into the apparatus.

FIG. 8 shows a configuration where the single-use container has a space 113 configured to receive the anvil 300. In this specific exemplary aspect, a portion of the anvil 306 is configured to be inserted into space 113 and unseal the first chamber 102. In certain aspects, the portion of the anvil 306 can also be heated.

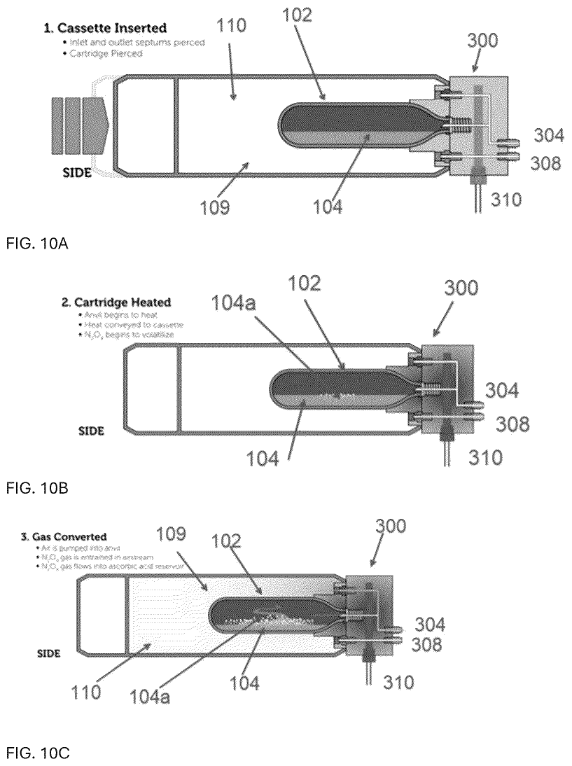

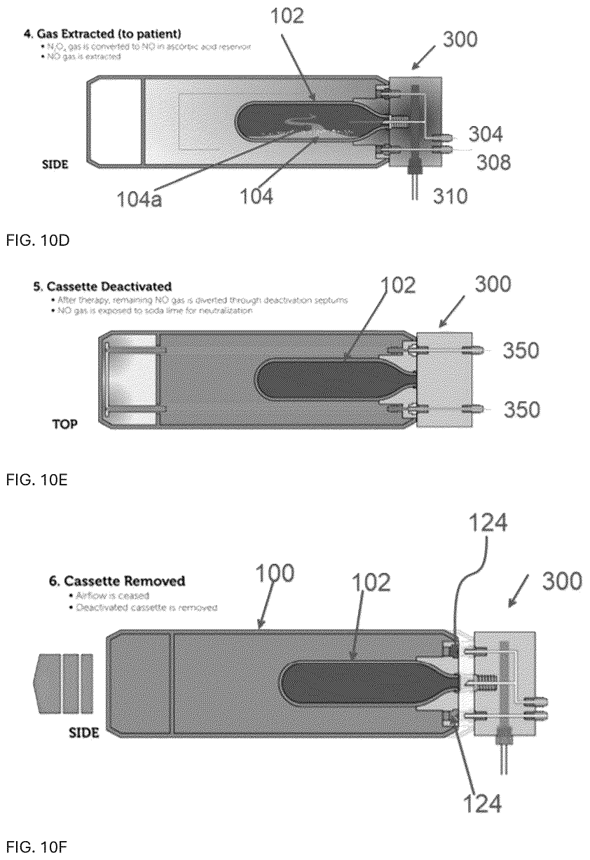

In FIGS. 9-10F, the proximal end 111 of the single-use container 100, is engaged with the anvil 300.

The single-use container is depicted in FIGS. 13A-19, when inserted into the apparatus, can also be engaged with a mating surface of the apparatus (not shown). Such a surface can be a portion of an additional anvil disposed within the apparatus or any other surface that can comprise mating elements that can engage with the single-use container.

In still further aspects, the apparatus is configured to activate the single-use container to form the nitric oxide.

It is understood that the way the single-use container is activated would depend on the configuration of the single-use container and the apparatus.

For example, as shown in FIG. 1, when anvil 300 is engaged with the single-use container 100, the proximal edge 107 interacts with one or more elements 303 of the anvil. Each of the one or more elements 303 comprises a lumen configured to create a fluidic path between the apparatus and the single-use container. In such aspects, one or more of the elements 303 are configured to unseal at least a portion of the first and/or second chambers. It is understood, however, that the unsealing occurs at a specific location by insertion of the one or more elements 303 into the designated chamber. The unsealing occurs between the chamber and the one or more elements but not between the chamber and the surrounding environment. In other words, the term unsealing, as used herein, refers to creating a fluidic path between the first and the second chambers and the apparatus using the lumens of the one or more elements 303a-303b. It is understood that the mere insertion of the one or more elements into the single-use container does not unseal the container itself to the surroundings. In fact, in some exemplary aspects, one or more elements are configured such that when they are inserted into the first and/or the second chambers, they form an additional seal between the chambers and the surrounding environment.

In still further aspects, the lumen of the one or more elements 303a-303b is configured to transport a gas to and from the desired chambers. For example, and without limitations, one or more of the 303a-303b units can deliver a gas mixture 302 from the apparatus to the first chamber. In such exemplary aspects, the gas mixture can comprise air, oxygen-enriched air, oxygen, nitrogen, or any combination thereof. The unsealing of the first chamber creates a fluid passage that allows the N₂O₄ present in the first chamber to be exposed to additional volume, enabling the liquid to undergo a phase change from liquid to gas and formation of NO₂ for conveyance 304 to the second chamber 109 comprising the antioxidant 110. The flow 304 can comprise a gas mixture delivered to the first chamber and the NO₂.

It is understood that the NO₂ formation process and the rate at which NO₂ flows through the first fluid passage can be controlled through the heating or cooling of the mating surfaces, which in turn heats or cools the N₂O₄/NO₂ mixture. Controlling the temperature can also allow control of the amount of NO₂ formed and, as a result, control the amount of NO formed in the single-use container. It is understood, and as described in this disclosure, the cooling, if present, can be achieved by any known in the art methods. For example, the cooling system can comprise fins, fans, and the like. In such exemplary and unlimited aspects, the fins and a fan can be activated to blow air across the fins. Yet, in other aspects, a Peltier-type cooling to extract heat from the process can be utilized.

In certain aspects, additional valves may be present to open and close communication between the N₂O₄ portion of the container and the one or more elements 303a-303b. Opening and closing of these valves enable control of the conveyance of NO2 through the lumens. Once NO₂ is mixed into the gas mixture 302, the mixed flow passes into the antioxidant portion of the container. As the NO₂ gas mixture passes through the antioxidant, it reacts in a manner that converts NO₂ to NO through a series of reactions gas. The formed NO gas is then collected through lumen 303c and delivered as a NO mixture 308 to a patient. It is understood that in these exemplary aspects, the NO is conveyed to the patient as a mixture of NO and conveying gas described herein, wherein conveying gas can comprise air, oxygen-enriched air, nitrogen, or any combination thereof.

The concentration of NO within the mixed flow 308, which exits the antioxidant, is determined by the outflow rate of NO₂ from the first chamber, which initially contains liquid N₂O₄, as well as the flow rate and components of the conveyance gas 302, with which the NO2 mixes.

FIG. 2 shows additional aspects, where the anvil 300 can push the first chamber 102 through the foil 214 into a secondary chamber 102a. Yet, in other aspects, only liquid N₂O₄ is pushed to the secondary chamber 102a. In this exemplary configuration, the conveyance gases are provided from the distal end of the single-use container. For example, the gas mixture 206 and NO₂ 212 interact with the antioxidant in the second chamber to form NO gas mixture 208 that is delivered to a patient.

FIG. 3 shows an exemplary configuration, where the one element 303a unseals the first chamber 102 and delivers a gas mixture 304 to the first chamber. Still further, a NO₂ gas is released, forming a conveyance gas 305 that delivers it to the second chamber through the element 303b. The NO gas 308 formed as a reaction of NO₂ with antioxidants is then delivered through lumen 303c to a patient.

As described above, some of the configurations can comprise an anvil comprising a secondary first chamber 600 comprising a liquid N₂O₄ 602 (FIG. 6). In this configuration, a gas mixture 304 passes through the secondary first chamber 600. The NO₂ gas formed in this chamber is then mixed with the gas mixture 304 to form NO₂/gas mixture 305 that contacts the antioxidant material 110 in the second chamber of the single-use container. It can be seen that there is fluidic communication 307 between the first chamber 102 and the secondary first chamber 600. The formed NO gas mixture 308 is then delivered to the patient.

In FIG. 8, the gas mixture 304 is delivered through anvil 306 to the first chamber, and then the NO₂/gas mixture comes into contact with the antioxidant material. The formed NO gas mixture 308 is then delivered to the patient.

FIG. 9 shows the coupling schematic between the exemplary single-use container 100 and the anvil 300. When the anvil 300 engages with the proximal end 111 of the container, the piercing element 306 unseals the first chamber 102, while the piercing element 306a unseals the activation septum 124. It is understood that in the exemplary aspects disclosed herein, under a spring force, the single-use container proximal surface comes into substantially intimate contact with the anvil to ensure efficient conductive heat transfer.

The gas mixture 304 is delivered through lumens for the piercing elements to the first chamber. The NO₂/gas mixture is then delivered to the second chamber 109 comprising an antioxidant 110. The formed NO gas mixture 308 is then conveyed to the patient. In certain aspects, the anvil portion of the apparatus is heated. The distal end of the container, an inerting chamber 108 comprising inerting material, is positioned to inactivate N₂O₄ and/or NO₂ in case of a leak or when the remaining N₂O₄ and/or NO₂ are left in the system at the end of the procedure. In certain exemplary and unlimiting aspects, the single-use container can also comprise a deactivation plate. In such exemplary and unlimiting aspects, the deactivation plate is configured to distribute the flow coming from the proximal end of the cassette to the inerting chamber.

FIGS. 10A-10F show various steps of use of the single-use container. In FIG. 10A, the single-use container 100 and anvil 300 are engaged, and the seals are pierced. The anvil is then heated with the use of heating element 310 (FIG. 10B), and NO₂ (104a) starts forming from the liquid N₂O₄ (104). The gas mixture 304 is delivered to the first chamber and is transferred to the second chamber 109 together with NO₂ (FIG. 10C). The formed NO (from the interaction of NO₂ with the antioxidant in the second chamber) gas mixture 308 is then delivered to the patient (FIG. 10D). At the end, the first chamber is substantially free of liquid N₂O₄ and NO₂. Whatever remaining amounts of N₂O₄ and NO₂ that are potentially still left in the system can be inactivated by the inerting material and dumping plate (FIG. 10E). FIG. 10F shows the disengagement of the single-use container from the anvil.

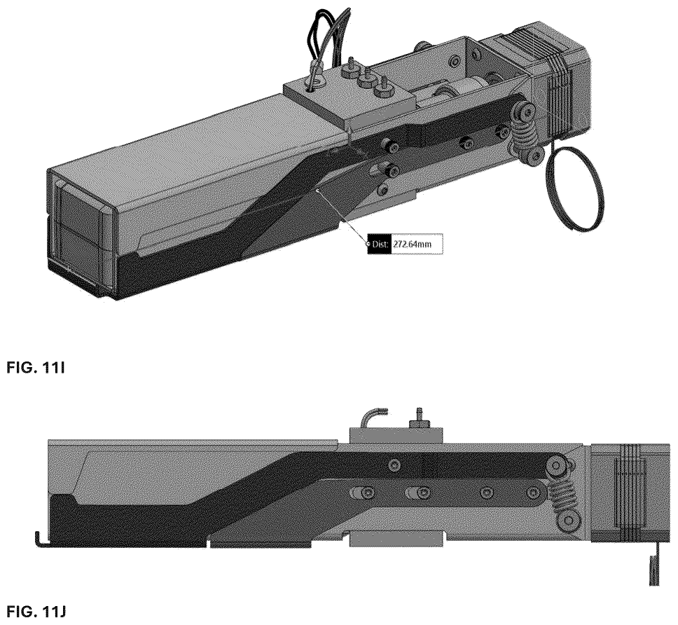

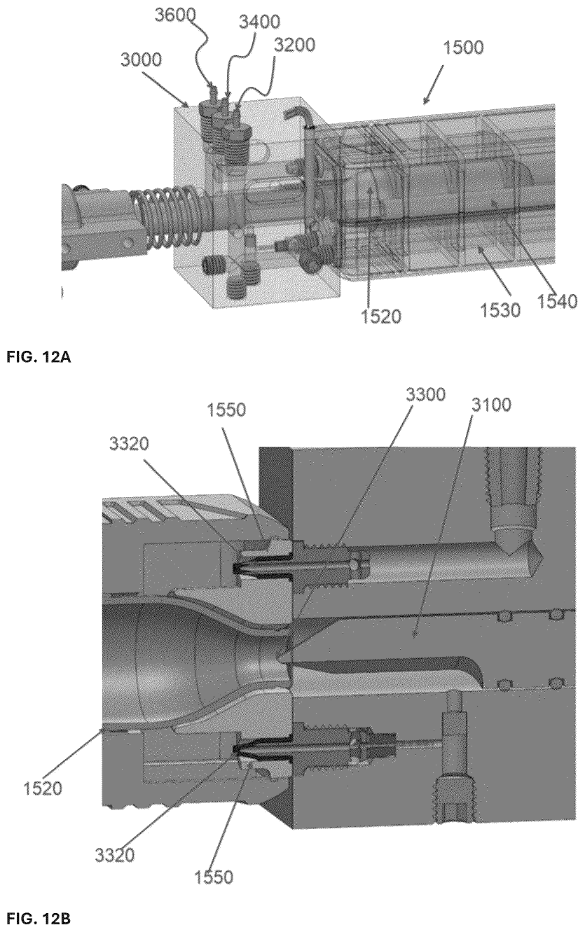

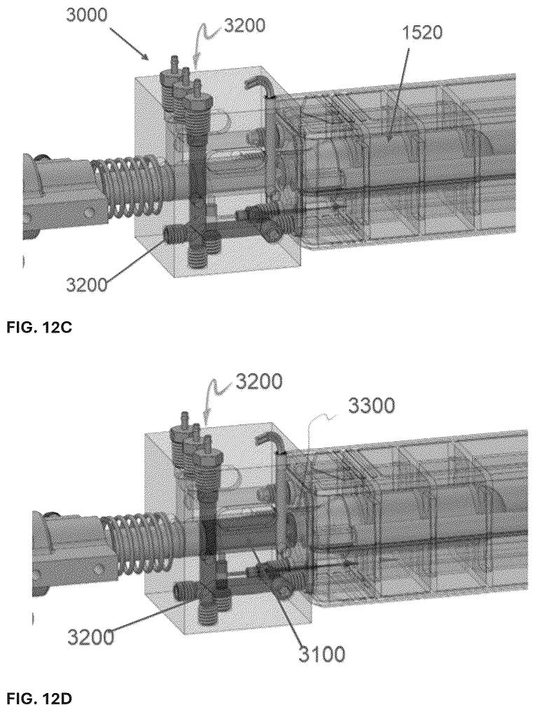

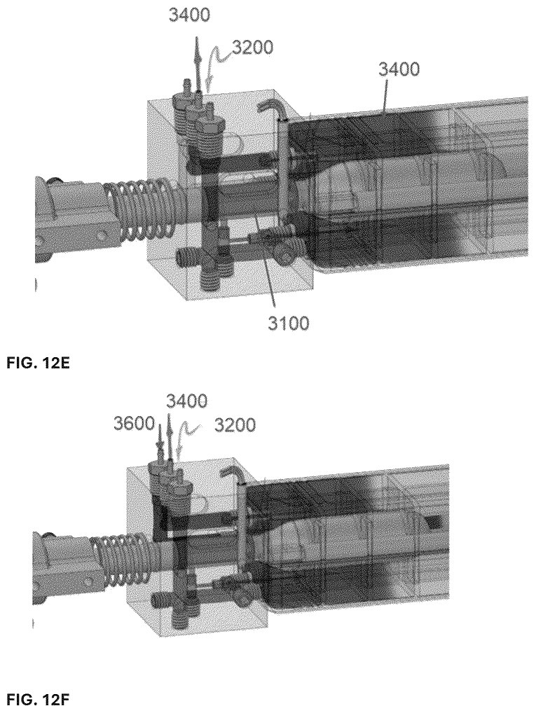

Additional aspects of the engagement between the anvil and the single-use container are shown in FIGS. 12A-12F. It can be seen that the single-use container 1550 comprises a first chamber 1520, the second chamber 1530, and the recycling tubes 1540. The anvil 3000 comprises fluidic paths 3200, 3400, and 3600 that can be utilized in various steps of the procedure. Upon engagement of the first chamber 1520 with the anvil, a firing pin 3300 present in the unsealing element 3100 unseals the first chamber while piercing elements 3320 unseal the activation septum 1550 (FIG. 12B).

The gas mixture 3200 starts immediately flowing through the system, and when the first chamber is unsealed, NO₂ gas 3100 gets mixed with the gas mixture 3200 (FIG. 12C) to arrive at the second chamber. The formed NO gas, then 3400, is then conveyed to the patient. (FIG. 12E).

It is understood that the system that comprises the apparatus and the single-use container can comprise a plurality of valves configured to control the gas flow as desired. It is also understood that the system can also comprise any fittings and tubing that would provide for the desired flow rate and desired flow direction. In certain aspects, the dimensions of the tubing can be determined based on the desired flow. In certain aspects, the tubing is a capillary tube. In still further aspects, the system can comprise O-rings and any other fitting and sealing elements as needed.

To deactivate the unit, a downstream valve can be used to redirect the remaining gas 3600 back into the single-unit recycling (dump) chamber (FIG. 12F).

Referencing back to FIGS. 12A-19. When the single-use container 1000 is inserted into the apparatus (not shown), valve 1130 is activated such that the first chamber is unsealed and a flow path between the first chamber and the second chamber is formed. In still further aspects, when the single-use container is positioned within the receptacle, at least a portion of the receiving surface of the receptacle mates with at least a portion of the proximal edge of the housing such that the second chamber is unsealed at a second chamber inlet 1060 and a second chamber outlet 1080.

In still further aspects, the fluid communication between the first chamber and the second chamber is interruptible. It is understood that the single-use container can comprise one or more valves configured to interrupt the fluid communication between the first chamber and the second chamber.

In still further aspects, at least the first chamber is thermally conductive or inductively heated. In yet still further aspects, at least a portion of the anvil is thermally conductive or inductively heated. In yet still further aspects, at least the first chamber is cooled. In yet still further aspects, at least a portion of the anvil is cooled.

In still further aspects, at least a portion of the single-use container is recyclable. It is understood that any portions of the single-use container can be recycled. For example, the flow-directing unit can be recyclable. In yet other aspects, the first chamber material can be recyclable.

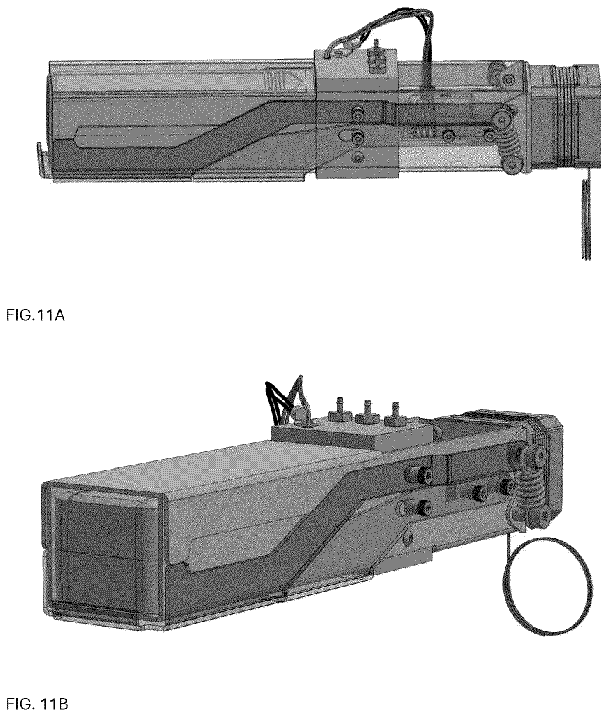

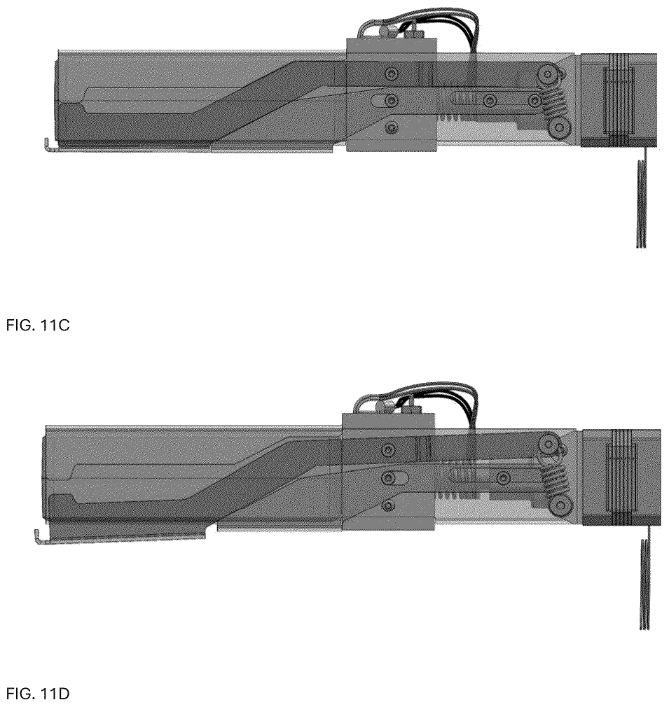

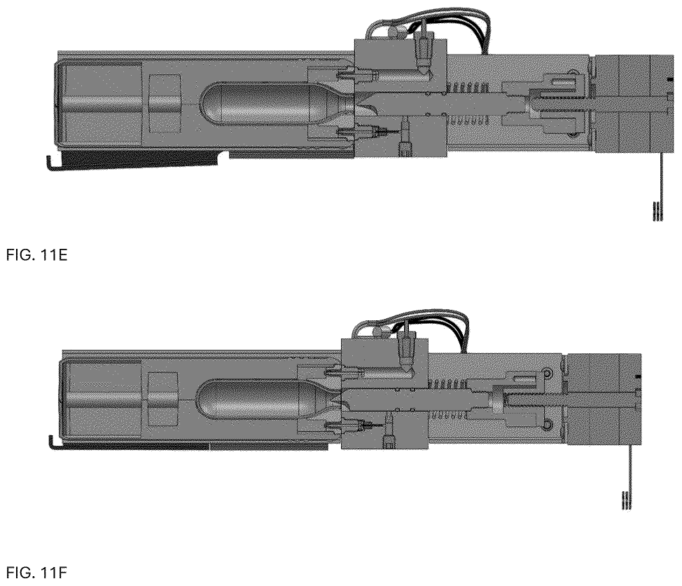

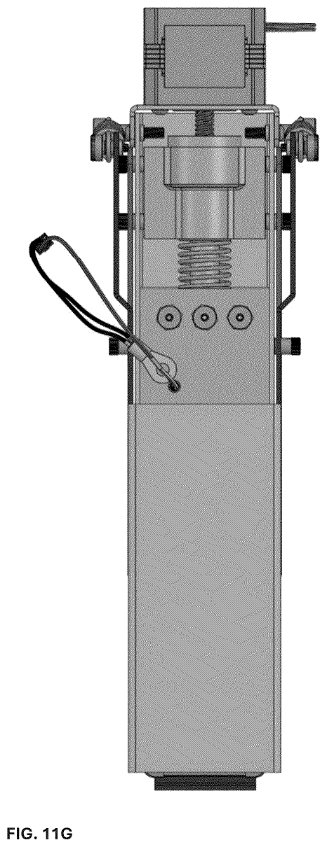

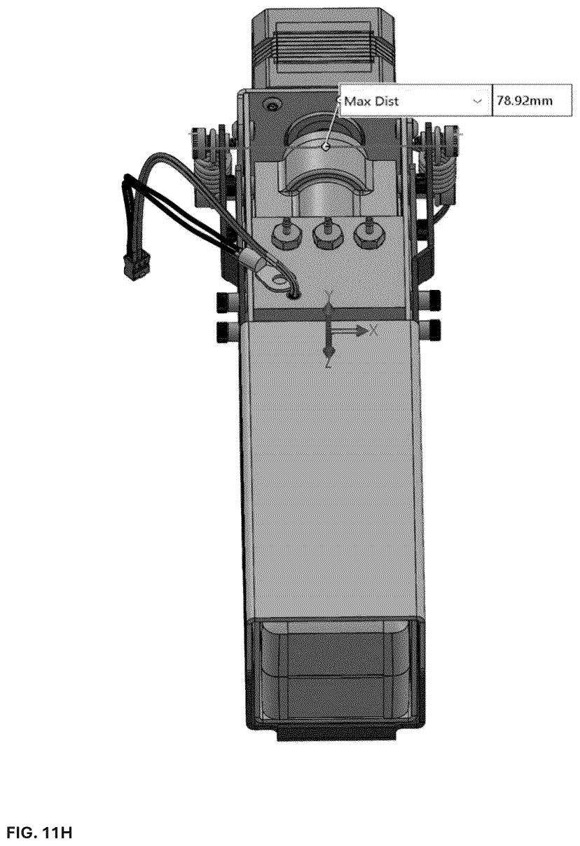

In still further aspects, the single-use container further comprises a locking mechanism configured to lock the single-use container within the apparatus. Such exemplary and unlimiting mechanisms are shown in FIGS. 11A-11J.

In still further aspects, the housing of the single-use container can further comprise a cooling element. In such exemplary and unlimiting aspects, at least the first chamber is in fluid communication with a cooling element. Yet, in other aspects, the cooling element can be positioned within the flow-directing unit. While in still other aspects, the cooling element can be positioned within the apparatus.

In still further aspects, the apparatus can comprise a control unit. In such exemplary and unlimiting aspects, the control unit is configured to engage the anvil with the single-use container.

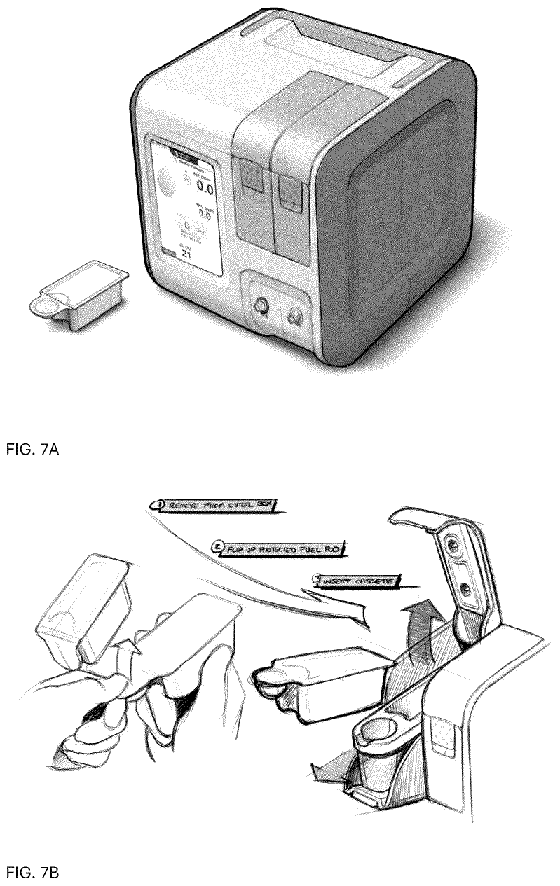

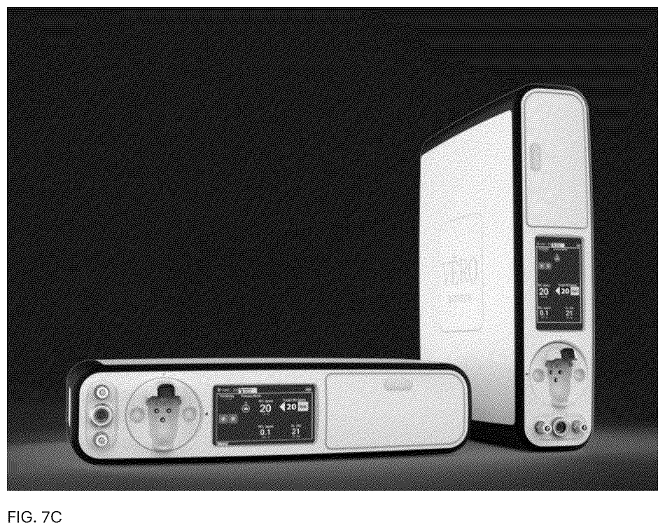

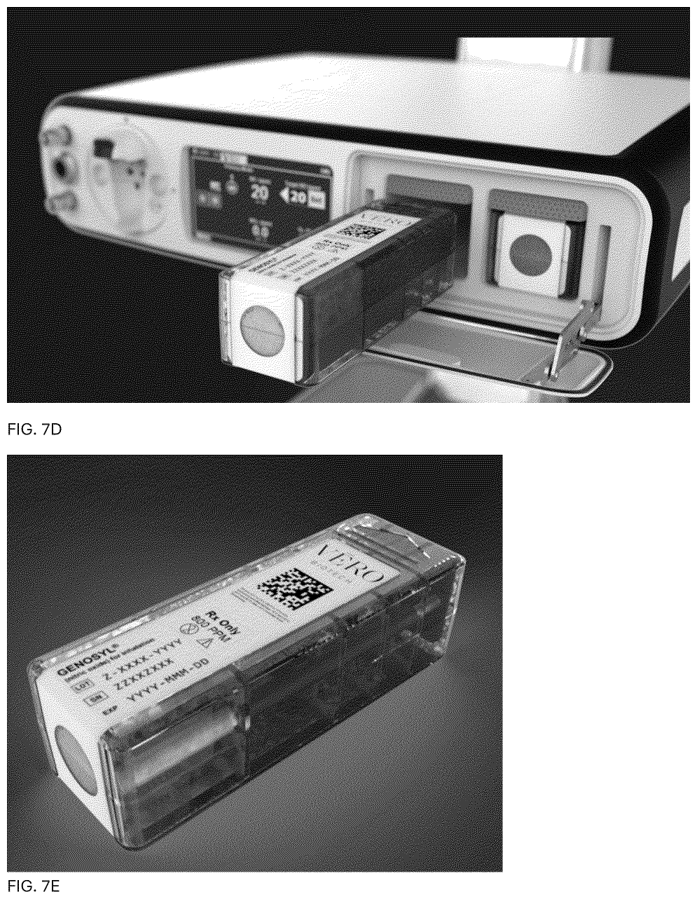

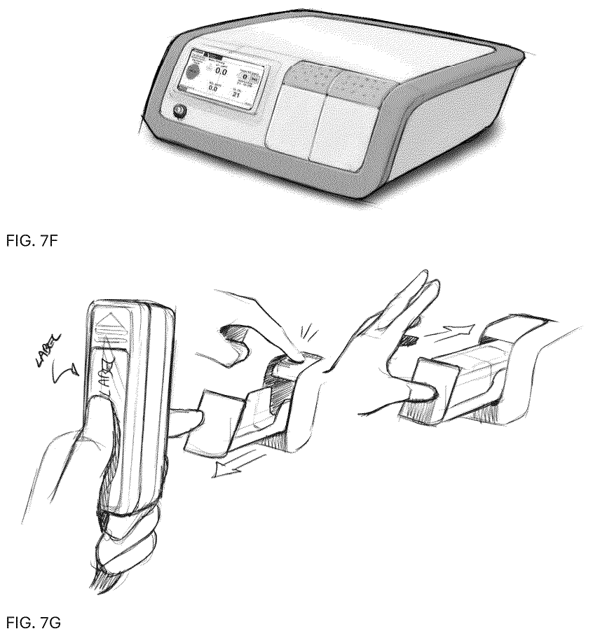

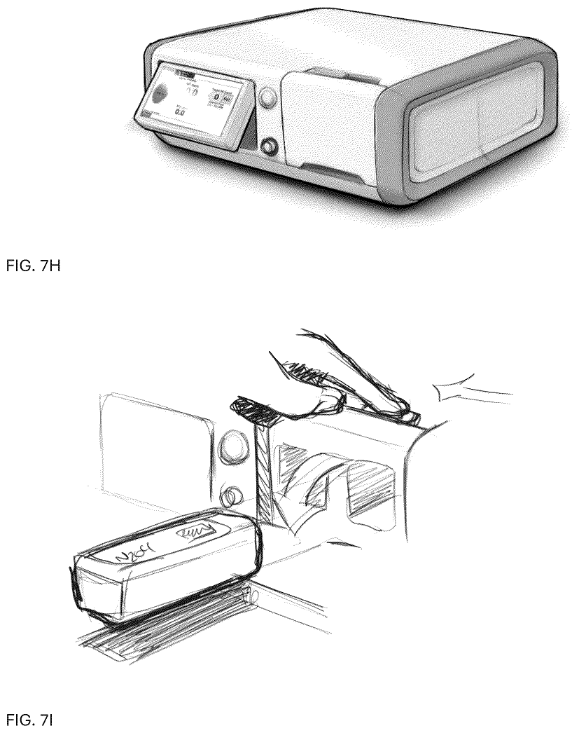

Some exemplary and unlimiting configurations of the single-use containers and apparatuses are shown in FIGS. 7A-7I.

Additional exemplary single-use containers are shown in FIGS. 20A-35 and are described in detail herein.

FIGS. 20A-20C show portions of an exemplary single-use container 2000. More specifically, the figures show exemplary designs for the first chamber in 2010 and the flow-directing unit in 2020.

The first chamber in this example, similar to the examples provided above and below, is configured to be directly filled with N₂O₄ or capable of caring for an ampule comprising N₂O₄. In such aspects, if the chamber is directly filled with N₂O₄, it can be sealed by any known in the art methods. For example, and without limitations, it can be sealed with a metal burst disc. If the chamber contains an ampule, the ampule is conventionally sealed. It is understood that the ampule can be made of glass or any other appropriate material that can withstand the operational conditions of the single-use container.

The first chamber, as described above, in certain aspects, can be constructed of stainless steel, aluminum, a metal alloy, or other thermally conductive metal to enable heating of the N₂O₄/NO₂. Yet in other aspects, the first chamber can be constructed of an inductive metal, which enables inductive heating of the N₂O₄/NO₂. Still, in further aspects, the first chamber can be constructed of a polymer or other suitable material to contain pressure wherein the material is thermally conductive (e.g., thermally conductive polymer). Still, in further aspects, the first chamber can be constructed of any non-conductive or insulating material but contain an inductive target inside, which is in contact with the N₂O₄ and can be heated inductively through the first chamber walls and subsequently transfer its heat to N₂O₄.

FIG. 20A shows the flow-directing unit 2020, having a valve, for example, a spool valve, 2021, positioned within a bed (not shown), similar to the one shown in FIG. 14A (1030). In still further aspects, the bed is substantially smooth to minimize the friction between the valve and the bed.

The distal end of the spool valve 2021 is defined by the sharp edge of a needle 2023. Any known in the art needles capable of unsealing the first chamber and/or ampule can be used. Some exemplary configurations of the needles are shown in FIG. 24. In certain aspects, the needle is a beveled needle. Yet, in other aspects, the needle comprises a least one orifice serving as a vent. In still further aspects, the at least one orifice is positioned on the side of the needle. In such an aspect, the orifice can provide a second flow path through the needle in case the flap covers the main opening in the tip after piercing. It also allows gas to escape to provide a more uniform and repeatable piercing.

In this exemplary aspect, the first chamber comprises a burst disc 2025 that can be unsealed by the described needle. It was found that the positioning of the burst disc within the flow-directing unit and not between the flow-directing unit and the first chamber is beneficial for the device's operation. The flow-directing unit receives the first chamber within the receiving portion 2080 and allows a more convenient filling and sealing of the first chamber, for example, similar to screwing on the cap 2090. It is understood that in such exemplary aspects, the single-use container may comprise an additional internal element ensuring a seal between the burst disc and an internal surface of the flow-directing unit.

In still further aspects, the flow-directing unit 2020 comprises an inlet 2022 and an outlet 2024 that are configured to mate with the appropriate portions of the anvil and are unsealed when the single-use container is inserted into the apparatus. The inlet 2022 and outlet 2024 can comprise a valve 2026 that helps direct the gas flow from the apparatus to the single-use container and from the single-use container to the apparatus. In certain aspects, the valve can be a duckbill valve.

In still further aspects, the flow-directing unit can comprise a micro-orifice plate 2027 similar to the one disclosed above. FIG. 20B shows a side perspective of the cross-section shown in FIG. 20A. FIG. 20C shows a close perspective of the first chamber and the flow-directing until the assembly. The outer shape of the first chamber and the flow-directing unit is substantially symmetrical, which makes it easier to seal the flow-directing unit within the outer body halves and to separate the second and inerting chambers. This simple shape also makes it possible to produce just one molded polymer matrix component and component and install two copies.

FIGS. 21A-21B show a schematic view of the single-use container 2010 containing the first chamber 2110 and the flow-directing unit 2130, similar to those shown in FIGS. 20A-20C. FIG. 21A shows a view of the single-use container with a top portion being removed. The single-use container 2100 further comprises the second chamber 2120 and the inerting chamber 2140. The darker shade (here is a blue shade) lines represent sealing faces between the top (not shown) and the bottom 2190 (FIG. 21B) portions of the outer housing. When the single-use container is activated to form NO from N₂O₄, the N₂O₄ and/or NO₂ flow is contained within the first and the second chambers. However, when N₂O₄ and/or NO₂ are diverted to the inerting flow paths, it flows through the channels on either side of the enclosure and into the inerting chamber 2140. In certain aspects, additional flow-directing elements can be positioned within the second chamber. Such flow-directing elements can comprise, for example, baffles that help in directing the gas flow during the formation of nitric oxide (NO).

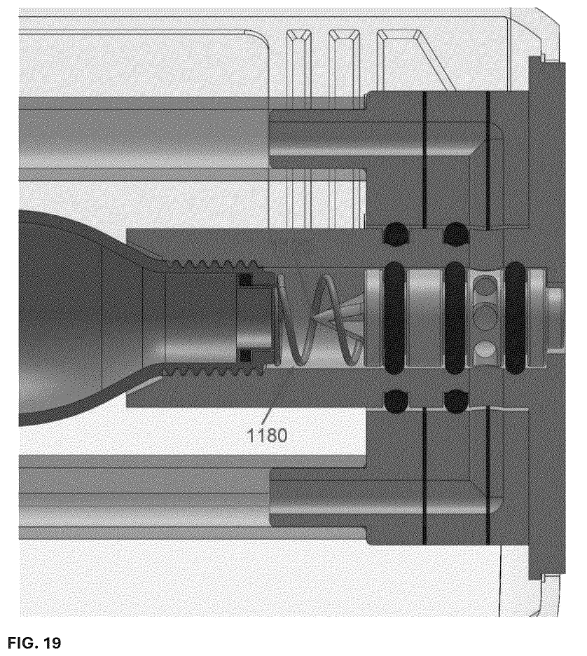

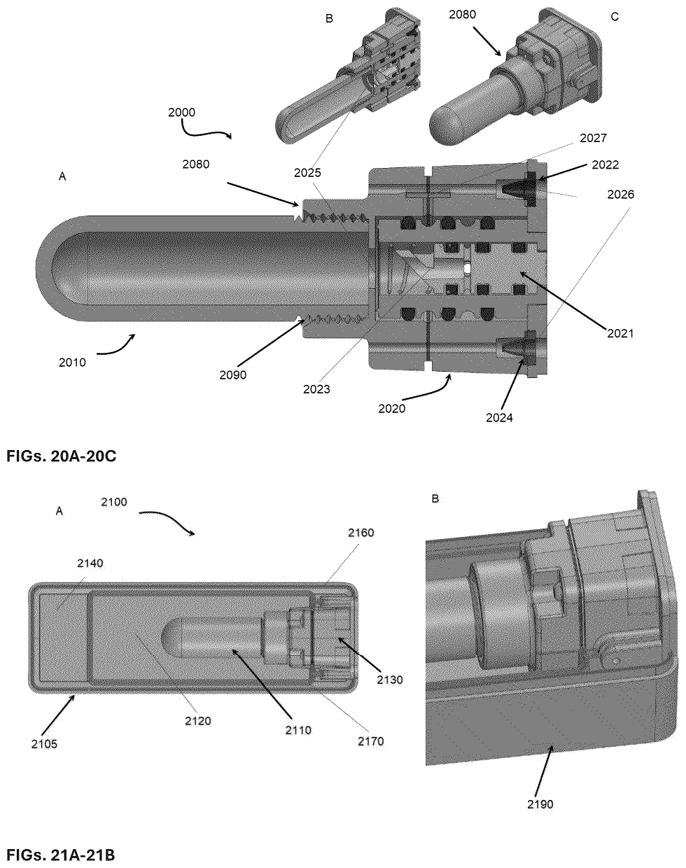

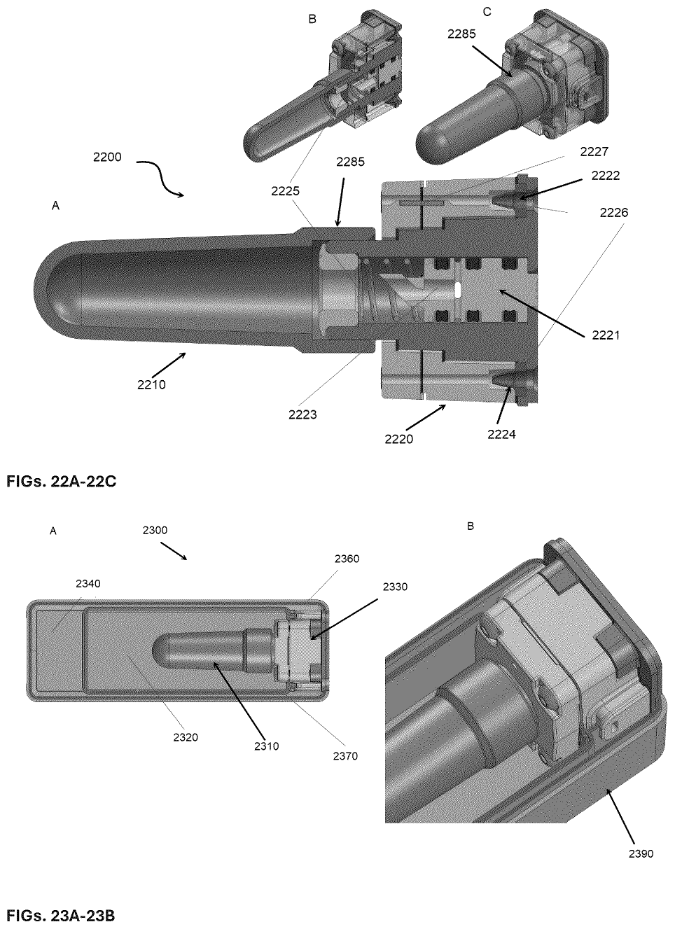

Referring to FIGS. 22A-22C. These figures describe an additional exemplary aspect of the current disclosure. The main components of the configuration shown in FIGS. 22A-22C are similar to those shown in FIGS. 20A-20C: the first chamber 2210, the flow-directing unit in 2220, the needle 2223, the inlet 2222, the outlet 2224, (where the inlet and outlet can comprise a valve 2226, such as a duckbill or other resealable valve. The burst disc 2225 is contained within the flow-directing unit but can retained distally by a threaded cap 2285 (that may double as a seal).

It is understood that the valve, for example, 2021, 2221, or any described herein valves within the flow-directing unit, can have a diameter that is suitable for the desired application. It is understood that in some aspects, the diameter of the valve, such as a spool valve, can determine the type and strength of the spring that holds this valve and thus affect the general activation and retraction mechanisms of the valve.

It is understood that any of the configurations disclosed herein without limitations can also comprise additional elements, such as example, O-rings, gaskets, valves, baffles, etc., to ensure better sealings, flow distribution, and the like.

FIGS. 23A-23B shows the views of the single-use container 2300 comprising elements shown in FIGS. 22A-22C. FIGS. 23A-23B show the first chamber 2310, and the flow-directing unit 2330. FIG. 23A shows a view of the single-use container with a top portion being removed. The single-use container 2300 further comprises the second chamber 2320 and the inerting chamber 2340. The darker shade (here is a blue shade) lines represent sealing faces between the top (not shown) and the bottom 2390 (FIG. 23B) portions of the outer housing. When the single-use container is activated to form NO from N₂O₄, the N₂O₄ and/or NO₂ flow is contained within the first and the second chambers. However, when N₂O₄ and/or NO₂ are diverted to the inerting flow paths, it flows through the channels on either side of the enclosure and into the inerting chamber 2140. In certain aspects, additional flow-directing elements can be positioned within the second chamber. Such flow-directing elements can comprise, for example, baffles that help in directing the gas flow during the formation of nitric oxide (NO).

FIG. 23C shows the single-use container 2300, similar to those shown in FIGS. 22A-22C and 23A-23B, wherein the second chamber 2322 contains any of the disclosed antioxidant-containing media and the first chamber 2310 contains, for example, liquid N₂O₄ 2315 and a gas space 2317. In aspects where the burst disc is pierced, some of the liquid N₂O₄ can be disposed of on the other side of the burst disc.

Referring to FIGS. 25A-25C. These figures describe an additional exemplary aspect of the current disclosure. The main components of the configuration shown in FIGS. 25A-25C are similar to those shown in FIGS. 20A-20C and 22A-22C: the first chamber 2510, the flow-directing unit in 2520, the needle 2523, the inlet 2522, the outlet 2524, (where the inlet and outlet can comprise a valve 2526, such as a duckbill valve. The burst disc 2525 is contained within the flow-directing unit but can retained distally by a threaded cap 2585 (that may double as a seal).

Similarly to other configurations described herein, the outer portions of the flow-directing unit can be produced from any known in the art materials. For example, it can be made from metal, plastic materials, or any combination thereof.

Further, in this embodiment, the press-fit cap that secures the burst disc and spool has a sufficiently small inner diameter to accept a first chamber with an externally threaded end.

FIGS. 26A-26B shows the views of the single-use container 2600 comprising elements shown in FIGS. 25A-25C. FIGS. 26A-26B show the first chamber 2610, and the flow-directing unit 2630. FIG. 26A shows a view of the single-use container with a top portion being removed. The single-use container 2600 further comprises the second chamber 2620 and the inerting chamber 2640. The darker shade (here is a blue shade) lines represent sealing faces between the top (not shown) and the bottom 2690 (FIG. 26B) portions of the outer housing. When the single-use container is activated to form NO from N₂O₄, the N₂O₄ flow is contained within the first and the second chambers. However, when N₂O₄ is diverted to the inerting flow paths, it flows through the channels on either side of the enclosure and into the inerting chamber 2140. In certain aspects, additional flow-directing elements can be positioned within the second chamber. Such flow-directing elements can comprise, for example, baffles that help in directing the gas flow during the formation of nitric oxide (NO).

FIG. 26C shows a general schematic of the single-use container 2600 shown in FIGS. 25A-26B, comprising a bottom portion of the housing 2690, a top portion 2694, the first chamber 2610, the second chamber 2620, the flow-directing unit 2630, and the inerting chamber 2640.

FIG. 26D shows a schematic assembly of the single-use container 2600. The bottom portion 2690 of the housing hosts the second chamber 2620, which is shown here schematically as comprising two-molded parts 2620a and 2620b comprising antioxidant-containing media. The molded antioxidant-containing media 2620a and 2620b can be made to include a space to receive the first chamber 2610. The top portion 2695 covers the housing. In certain aspects, the single-use container can further comprise a label or any other identifying element. It is understood that the labels or any other identifying elements can carry information for the caregiver or the patient with respect to content, use, dosage, precautions, and the like as they relate to the single-use container. The exemplary fully assembled single-use container is shown in the FIG. 26E.

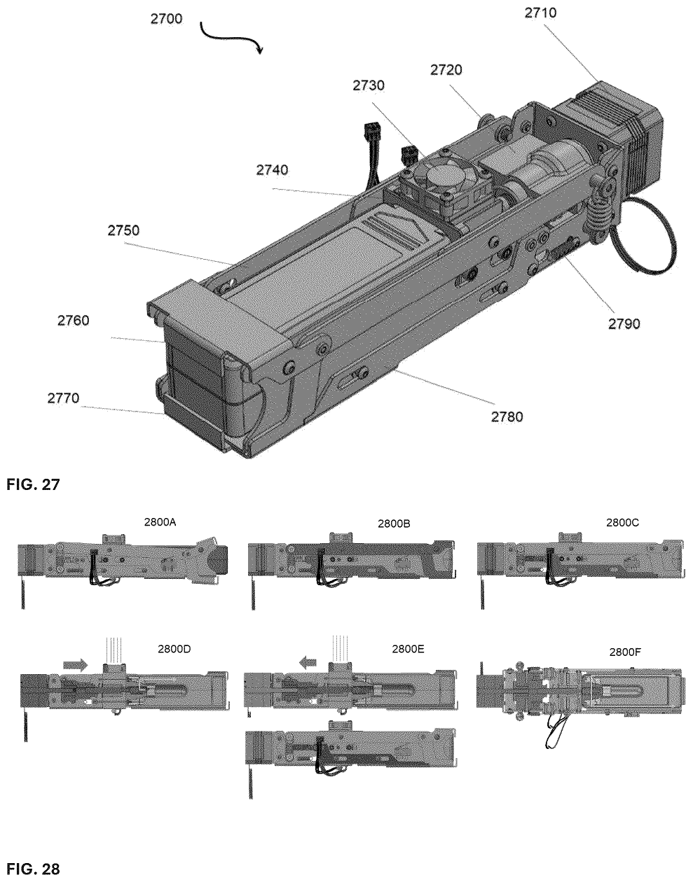

FIG. 27 shows the mechanism 2700 that can be used to activate the single-use container similar to the one shown in FIGS. 25A-26E.

Some exemplary portions of the mechanism are as follows. Heat can be transferred by contact between the anvil and the proximal face of the single-use container, which is also the proximal end of the flow-directing unit. The anvil is heated using a cartridge heater or other means, and the temperature is controlled through a feedback loop using a temperature probe such as a thermistor or thermocouple.

The anvil can be cooled using a fan that blows across or exhausts heat from heat transfer fins, which are part of the anvil. Cooling can also be achieved using a Peltier cooling or similar active cooling device placed in contact with the anvil.

The anvil can contain two valve-piercing or valve-opening elements, which, upon insertion of the single-use container into the mechanism, open flow paths for gas flow into the single-use container and for gas flow out of the single-use container. This is achieved by the elements either opening duck-bill or similar one-way type valves upon insertion, or by the elements having sharp needle end features that pierce a seal. The conveying gas flow into the single-use container can comprise air, oxygen-enriched air, nitrogen, or a combination thereof, and the flow out of the single-use container contains this conveying gas and the formed nitric oxide.

In still further aspects, the mechanism contains a firing pin that contacts and translates a valve with the needle end and side port to different positions to open and close fluid paths as well as piercing a burst disc (or a foil) or break a glass ampule to unseal the first chamber or unseal an ampule containing N₂O₄ into the first chamber.

In yet still further aspects, the mechanism can secure the single-use container once inserted using a two-stage process—when the single-use container is inserted by the user, there are upper and lower jaws that secure it in place once it reaches a certain point of insertion—at this point, the single-use container can still be ejected by depressing the eject lever because it is not yet activated—once the user commands the unit to start dosing, a locking plate is advanced forward which then prevents the upper and lower jaws from being able to open. Once the user is done using the single-use container and commands an ejection, the unit will be cooled, and the firing pin retracts first to an inerting position where the first chamber is fluidically connected to the inerting chamber and then retracts to a home position, which allows the locking plate to return to its home position that allows the user to depress the eject lever and remove the single-use container.

Some of the disclosed above elements are shown in FIG. 27-28 or 31. This configuration utilizes a stepper motor 2710 configured to push both the heater block 2740 and a firing pin 2720 against and into the flow-directing unit (not shown). The heater block is heated and monitored with heating devices (not shown). The mechanism can further comprise a cooling fan 2730 designed to further control the temperature within the first chamber to provide more responsive dose control. Cooling of the heater block is aided by the presence of fins, which increase the available surface area for heat transfer.

The mechanism further can comprise a locking plate 2780, the single-use container lock upper jaw 2760 and lower jaw 2770 (configured to lock the single-use container), and the sheet metal chassis (that forms the structure of the mechanism to hold the single-use container) 2750.

FIG. 28 shows exemplary and unlimiting steps of activation of the single-use container. In step 2800A, the user inserts the single-use container into an apparatus, temporarily opening the sheet metal jaw and closing the limit switch (opening its circuit).

In step 2800B, the single-use container seats itself in the carriage against the heater block and allows the jaw to open the limit switch.

In step 2800C, the limit switch is open (circuit closed), and the stepper motor can drive the firing pin and locking plate forward until the locking plate is stopped by the lower jaw, preventing it from opening during therapy.

In step 2800D, which shows a section view, when the thermistor detects that the heater block has reached the target temperature, the stepper motor can be activated. It travels forward to puncture the burst disc, opens the path to the second chamber, and stops. The air begins to flow over the micro-orifice plate.

In step 2800E, when therapy is stopped, the heater is deactivated, and the fan is activated to cool the heater block. The stepper motor retracts partially to allow the spool valve to direct gas into the inerting chamber (step 2800F), but not far enough to allow the locking plate to slide clear of the lower jaws.

In step 2800F, which shows a top section view, when the firing pin is retracted, the spring returns the spool valve to its starting position, allowing the remaining gas to vent into the inerting chambers wrapped around both sides of the second chamber. Jaws are still locked until the inerting process is complete.

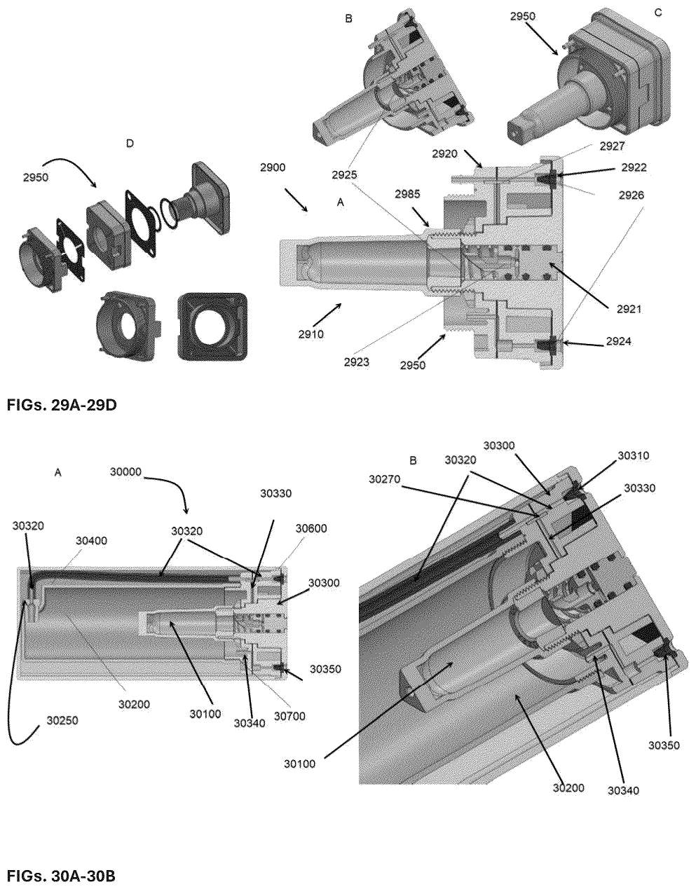

FIGS. 29A-29D show an additional configuration of the first chamber 2910 and the flow-direction unit 2920 that can be used in the single-use container 2900. These figures describe an additional exemplary aspect of the current disclosure. The main components of the configuration shown in FIGS. 29A-29C are similar to those shown in FIGS. 20A-20C, 22A-22C, and FIGS. 25A-25C: the first chamber 2910, the flow-directing unit in 2920, the needle 2923, the inlet 2922, the outlet 2924, (where the inlet and outlet can comprise a valve 2926, such as a Vernay Duckbill Valve. The burst disc 2925 is contained within the flow-directing unit but can retained distally by a threaded cap 2985 (that may double as a seal). An additional threading portion 2950 (the receiving portion for the second chamber to be screwed onto) is shown in FIGS. 29A-29D is directed to receive the second chamber, 30200.

FIGS. 30A-30B show the single-use container 30000 comprising the first chamber 30100 and the flow-directing unit 30300 as shown in FIGS. 29A-29D. 30600 and 30700 show outer and inner seals, respectively. As shown in FIG. 30B, the inlet 30310, and the outlet 30350 are in communication with the second chamber 30200. In this exemplary configuration, the inlet 30310 extends into a first fluidic path 30320 that is configured to deliver a conveying gas provided by the apparatus. It is understood that any of the disclosed above conveying gases can be used. For example, the conveying gas can comprise air, oxygen-enriched air, nitrogen, or any combination thereof.

In still further exemplary aspects, the valve can move such that the flow path 30330 between the first chamber and the second chamber is formed. This flow path delivers the gaseous nitrogen dioxide or the gaseous mixture of dinitrogen tetroxide N₂O₄ and nitrogen dioxide (NO₂) into the first fluidic path 30320 through the micro-orifice plate 30270. In still further aspects, the gaseous nitrogen dioxide, or the gaseous mixture of dinitrogen tetroxide N₂O₄ and nitrogen dioxide (NO₂) are mixed with the conveying gas from the apparatus in the first fluidic path 30320. As can be seen in FIG. 30A, the first fluidic path 30320 is external to the second chamber 30200 and enters the second chamber at a base of a distal portion 30250 of the second chamber. While the first fluidic path in this example is external to the second chamber, it is understood that the aspects where the first fluidic path is internal to the second chamber are also disclosed.

Further, the second chamber comprises a second fluidic path, wherein the second fluidic path 30340 is in flow communication with the outlet 30350 and is configured to deliver formed nitric oxide to the patient.

FIG. 30C shows a schematic of the positioning of the inerting chamber 30400, respectively, to the first chamber 30100, the second chamber 30200, and the flow-directing unit 30300. FIG. 30D shows the assembly of the second chamber 30200, the first chamber 30100, and the flow-directing unit 30300 into the housing 30900.

FIG. 31 shows a mechanism 31000 that can be used to activate the single-use container shown in FIGS. 29A-30D. The mechanism has a stepper motor 31100, firing pin adapter 31300, device heater block 31500, fan 31400, sheet metal chassis 31700, locking plate 31800, single-use container lock spring 31200, locking plate return spring 31600, single-use container lock upper jaw 31200 and lock upper shoe 31300, single-use container lock lower jaw 31100, single-use container receiver tunnel 31900 and eject lever 31400.

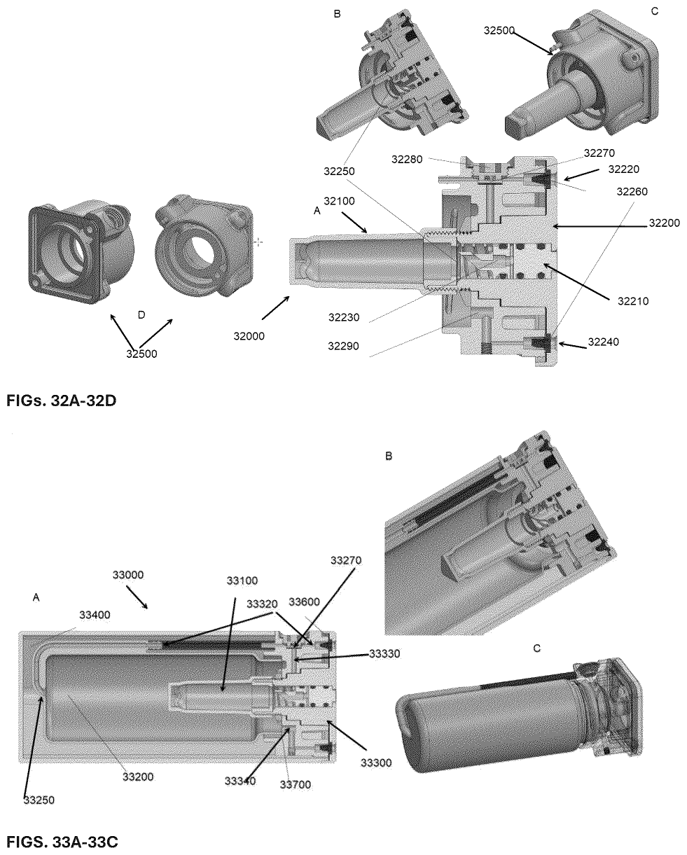

FIGS. 32A-32D show additional exemplary configurations of the first chamber 32100 and the flow-direction unit 32200 that can be used in the single-use container 32000. These figures describe an additional exemplary aspect of the current disclosure. The main components of the configuration shown in FIGS. 32A-32C are similar to those shown in FIGS. 20A-20C, 22A-22C, FIGS. 25A-25C, and FIGS. 29A-29C: the first chamber 32100, the flow-directing unit in 32200, the needle 32230, the inlet 32220, the outlet 32240, (where the inlet and outlet can comprise a valve 32260, such as a duckbill valve. The burst disc 32250 is contained within the flow-directing unit. The flow-directing unit can also comprise an outlet frit receptacle 32390. An additional threading portion 32500, as shown in FIGS. 32A-32D is directed to receive the second chamber 33200.

FIGS. 33A-33C show the single-use container 33000 comprising the first chamber and the flow-directing unit shown in FIGS. 32A-32D. In still further exemplary aspects, the valve can move such that the flow path 33330 between the first chamber 33100 and the second chamber 33200 is formed. This flow path delivers the gaseous nitrogen dioxide or the gaseous mixture of dinitrogen tetroxide N₂O₄ and nitrogen dioxide (NO₂) into the first fluidic path 33320 through the micro-orifice plate 33270. In still further aspects, the gaseous nitrogen dioxide, or the gaseous mixture of dinitrogen tetroxide N₂O₄ and nitrogen dioxide (NO₂), is mixed with the conveying gas from the apparatus in the first fluidic path 33320. As can be seen in FIG. 33A, the first fluidic path 33320 is external to the second chamber 33200 and enters the second chamber at a base of a distal portion 33250 of the second chamber.

In this example, the second chamber 33200 can be an extrusion blow-molded vessel (as shown, for example, in FIG. 33F). The inerting chamber 33400 can be formed by the cavity between the second chamber and the outer body of the single-use container.

Further, the second chamber comprises a second fluidic path, wherein the second fluidic path 33340 is in flow communication with the outlet and is configured to deliver formed nitric oxide to the patient.

FIG. 33B-33E show additional schematics of the single-use cell shown in FIG. 33A.

FIGS. 34A-34G show various configurations of the spool valve. The valves can include a steeply beveled needle tip with a side-venting port and an internal lumen. The beveled tip accommodates both foil/disc piercing and ampule shattering when the spool is translated down the axis of the single-use container during activation. The lumen within it connects openings on the face and side of the needle to a plurality of gas passages exiting perpendicular to the axis of the lumen. A small “frit” or filter can be installed in the lumen to prevent the ingestion of glass shards or fluid into these gas passages.

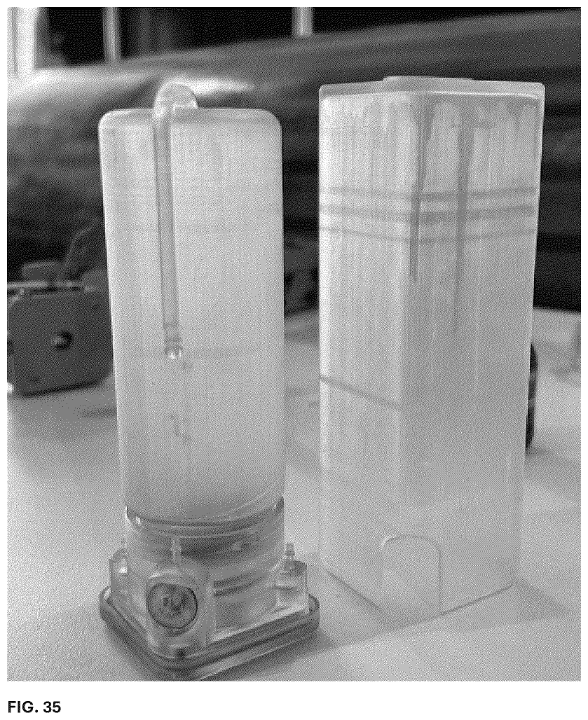

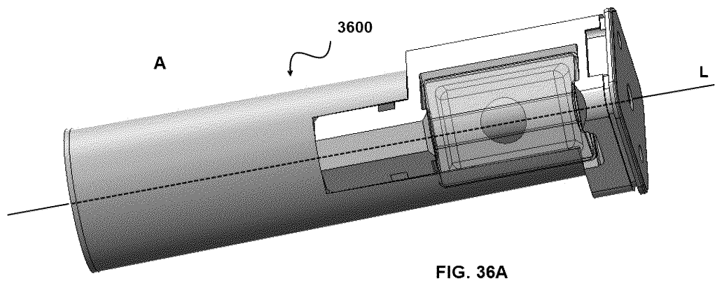

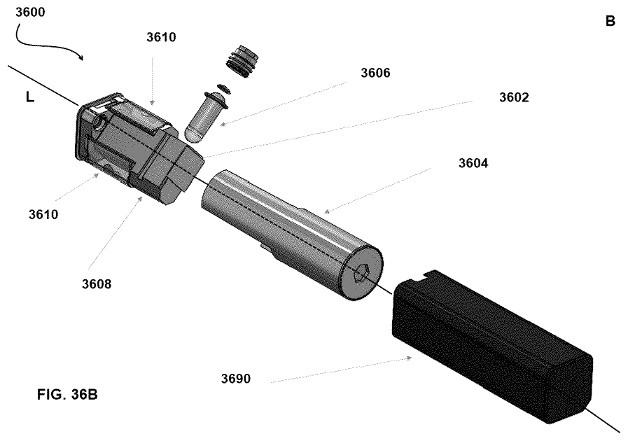

FIG. 35 shows an exemplary single-use container according to some aspects of this disclosure.

An additional aspects of the single-use containers are shown in FIGS. 36A-37B. As discussed above, FIG. 36A shows an assembled version of the exemplary single-use container 3600, where L represents the main longitudinal axis of the container. FIGS. 36B-36F show an exploded view of such a container. In these exemplary and unlimiting aspects, the first chamber 3602 is positioned within the flow-directing unit 3608 and is configured to host an ampule 3606. The ampule 3606 is positioned perpendicular to the main longitudinal axis L (FIG. 36B). The first chamber and the flow-directing units are in communication with the second chamber 3604, and all the parts are hosted within the housing 3690. The two inerting chambers 3610 are positioned within the proximal portion of the single-use container and on the both sides of the flow-directing unit 3608.

FIGS. 36C and 36D show a further exploded view of the container, where more details of the flow-directing unit 3608 are provided. The ampule 3602 is inserted in the first chamber 3602 and is sealed from the surrounding environment by any sealing means 3630 that can include gaskets, O-rings, sealing rings, nuts, bolts, screws, etc. The flow-directing unit 3608 hosts a spool valve 3612, which operates with a spring 3616. Again, all the portions of the flow directing unit, if needed, have additional sealing members 3630 that include seals, O-rings, gaskets, etc. The distal portion of the valve 3612 comprises unsealing means 3614 that are configured to break the ampule 3606 when the container is activated. In certain aspects, the unsealing means 3614 can be releasably attached to the valve 3612. In certain exemplary and unlimiting aspects, when the ampule is unsealed, the unsealing means 3614 can be released from the valve 3612 and stay within the ampule body, thereby preventing undesirable release of broken ampule debris or other particulars to entry the gas flow paths.

In still further aspects, the flow-directing unit can further comprise one or more one-way valves 3640 that help direct the gas flow from the apparatus to the single-use container and from the single-use container to the apparatus. In certain aspects, these one-way valves can be duckbill valves. In still further aspects, these duckbill valves can help the flow-directing unit to direct the gas flow from an inlet to an outlet of the container, as further disclosed below.

In still further aspects, the flow-directing unit can comprise one or more orifices 3672 in which micro-orifices plates 3670 are inserted. Again, all the orifices and openings can be sealed as needed by sealing means 3630 as needed. In FIG. 36D the micro-orifice plates are installed in the holes 3672 and then sealed into place using O-rings and retaining screws (collectively called sealing means 3630). It can be further observed in FIG. 37A. The NO₂ that is formed from N₂O₄ in the first chamber then passes through these micro-orifices, where it is picked up by the conveying gas (blue arrow).

The second chamber 3604 of this exemplary single-use container is shown in FIG. 36F. The antioxidant material 3605 is positioned within the housing and is terminated with end cap 3607.

FIGS. 37A-37B show cutaway views of this exemplary single-use container, and they also show the flow gas paths formed in the container upon activation.

FIG. 37A shows the single-use container 3600 comprising the first chamber 3602 that is positioned within the flow-directing unit 3608. As shown in FIG. 37A, the inlet 3641, and the outlet 3643 are in communication with the second chamber 3604. In this exemplary configuration, the inlet 3641 extends into a first fluidic path 3621 that is configured to deliver a conveying gas provided by the apparatus. It is understood that any of the disclosed above conveying gases can be used. For example, the conveying gas can comprise air, oxygen-enriched air, nitrogen, or any combination thereof.

In still further exemplary aspects, the valve can move such that the flow path 3625 between the first chamber and the second chamber is formed. This flow path delivers the gaseous nitrogen dioxide or the gaseous mixture of dinitrogen tetroxide N₂O₄ and nitrogen dioxide (NO₂) into the first fluidic path 3621 through one or more micro-orifice plates 3670. In still further aspects, the gaseous nitrogen dioxide, or the gaseous mixture of dinitrogen tetroxide N₂O₄ and nitrogen dioxide (NO₂), is mixed with the conveying gas from the apparatus in the first fluidic path 3621. Further, the second chamber comprises a second fluidic path, wherein the second fluidic path 3623 is in flow communication with the outlet 3643 and is configured to deliver formed nitric oxide to the patient.

The one or more micro-orifice plates 3670 can have the same or different sizes. In certain aspects, the one or more micro-orifice plates 3670 have a different size that allows for a larger dosing range. The size of the one or more micro-orifices can be 1 micron to 100 microns, including exemplary values of 3, 5, 10, 15, 20, 25, 30, 40, 50, 60, 70, 80 and 90 microns. It is understood that any ranges between any two foregoing values can be formed. For example, and without limitations, the one or more orifices can have a size of 1 to 90 microns, 1 to 70 microns, 1 to 50 microns, 1 to 20 microns, 1 to 10 microns, 5 to 100 microns, 5 to 90 microns, 5 to 50 microns, 5 to 30 microns, and so on. The size of each orifice can assist in determining the flow of nitric oxide as desired. In such aspects, the flow-directing unit can be constructed such that it can accept orifice plates with different orifice sizes—this capability will create the opportunity for single-use containers that support specific dosage ranges—e.g., micro-dosing, standard, high-dose, etc.

In still further aspects, the spool valve 3612 can have various positions. In these exemplary and unlimiting aspects, the spool valve 3612 can have three positions, for example. In one position, the spool valve 3612 breaks the ampule 3606 with the unsealing means 3614 and aligns the gas flow from the ampule to the first micro-orifice plate 3670. In a second position, the valve is configured to align the flow with a second micro-orifice plate 3670. In yet a third position, the valve is configured to align the gas flow with one or more inerting chambers if needed.

In still further aspects, the second chamber 3604 can also comprise a septum 3609. Without wishing to be bound by any theory, it is understood that the septum 3609 is positioned such that it allows the desired direction of the gas flow, as shown with arrows.

FIG. 37B shows a top cutaway view of the single-use container. This view allows one to see the positioning of the one or more inerting chambers 3610 relative to other components (first and second chambers, ampule, valve, etc.) of the single-use container.

It is further understood that this exemplary single-use container can interact with the apparatus similarly to other single-use containers disclosed herein and described above.

It is further understood that the single-use container can be provided in different dosing versions. In such aspects, the different dosing versions of single-use containers can be color-coded and RFID identifiable to distinguish the dosing range and environments of use. Additional possible versions of the single-use container can include high-flow/high-dose and low-flow/low-dose containers, containers for cardiac catheterization labs, standard-dose containers, micro-dose containers, containers for use in neonatal, pediatric, or adult treatment, or containers to provide the treatment during transport of the patient (for example, in the ambulance, medical helicopter, or any other vehicle or aircraft).

In still further aspects, the different versions of the single-use container can be enabled by an interchangeable orifice plate assembly that accepts orifice plates with different hole sizes, allowing more or less nitrogen dioxide gas to exit the first chamber as described above.

In still further aspects, disclosed herein is an apparatus comprising: an anvil positioned within a receptacle of the apparatus, wherein the receptacle defines a space configured to receive the single-use container of any of the examples herein, wherein the anvil is engageable with at least the proximal edge of the single-use container, as described in detail above.

In still further aspects, the anvil comprises a first element configured to engage with a first chamber of the single-use container. In such exemplary aspects, the first element is a firing pin configured to unseal the first edge of the valve and to move the valve along the main axis of the single-use container such that the needle unseals the first chamber. In still further aspects, the first element is fluidically connected with an internal lumen of the valve, providing an internal fluidic path connecting the first chamber and the apparatus.

In some exemplary and unlimiting aspects, the anvil can comprise a second element configured to engage with the inlet of the flow-directing unit, such that a first fluidic path connecting the second chamber and the apparatus is formed. In yet still, in further aspects, the anvil can comprise a third element configured to engage with the outlet of the flow-directing unit, such that a second fluidic path connecting the second chamber and the apparatus is formed. In such exemplary and unlimiting aspects, the first fluidic path is directed from the apparatus to the second chamber, and the second fluidic path is directed from the second chamber to the apparatus.

In still further aspects, the apparatus comprises a heating element and/or cooling element. It is understood that the heating element and/or cooling element can be positioned in the anvil, the receptacle of the apparatus, or any combination thereof.

In still further aspects, wherein the internal, first, and/or second fluidic paths comprise a conveying gas comprising air, nitrogen, oxygen, or a combination thereof, wherein the conveying gas is supplied by the apparatus.

In still further aspects, the apparatus disclosed herein can further comprise a control unit. In such exemplary and unlimiting aspects, the control unit is configured to engage the anvil with the single-use container. In yet still further aspects, the anvil can further comprise a temperature measuring device that is in a feedback loop communication with the control unit. In still further aspects, the apparatus is configured to deliver nitric oxide to a subject.

Also disclosed herein are systems comprising: the single-use container of any of the examples herein, and the apparatus of any of the examples herein.

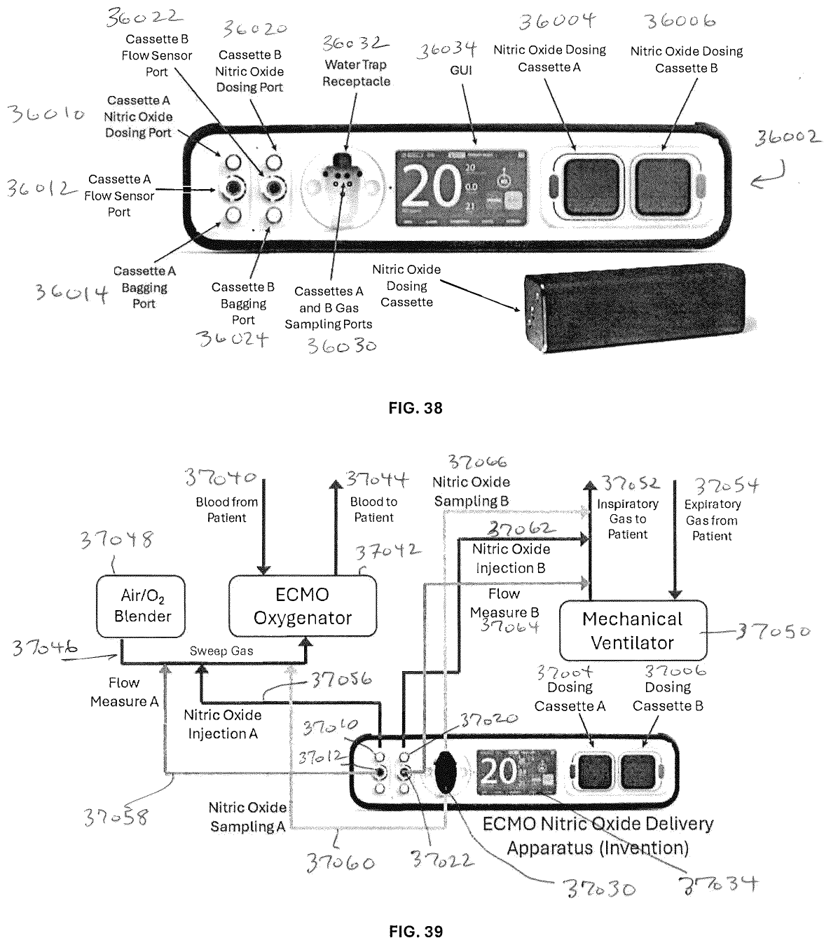

In some aspects, the apparatus for delivery of nitric oxide during ECMO treatment or, more generally, during cardiopulmonary bypass (CPB). In some aspects, the apparatus is used for the delivery of nitric oxide to an ECMO device and/or a ventilator. As shown in FIG. 38, a delivery apparatus 36002 can include nitric oxide dosing cassettes 36004 and 36006. The apparatus 36002 includes both physical and software components. The apparatus 36002 contains two completely independent nitric oxide dosing systems (A and B). The nitric acid dosage system A includes nitric oxide dosing cassette A 36004, nitric oxide dosing port 36010, flow sensor port 36012, and bagging port 36014. The nitric acid dosage system B includes nitric oxide dosing cassette B 36006, nitric oxide dosing port 36020, flow sensor port 36022, and bagging port 36024. In addition, the apparatus can include gas sampling ports (shown generally as 36030) for sampling the flow from nitric acid dosage system A and/or nitric acid dosage system B. The apparatus can further include a water trap receptacle 36032 and a graphical user interface (GUI) 36034. The apparatus can also include a control unit and optionally a processing unit that can communicate with the nitric oxide dosing cassettes 36004 and 36006, at least one air inlet (e.g., 1060 in FIG. 13B), the GUI 36034, and the various ports included in the apparatus. Nitric oxide delivery from each cassette or system can be governed by a feedback loop that is based on sampling the nitric oxide in the delivery circuit (or flow) using gas sampling ports 36030 and adjusting the nitric oxide produced by the cassette or system based on a set nitric oxide value. This is a primary feedback loop. A flow rate measurement using flow sensor 36012 and/or flow sensor 36022 in the delivery circuit for each system can be used to provide additional feedback for nitric oxide delivery, providing a gauge of how much nitric oxide needs to be added based on the amount of dilution gas flowing in the circuit and the target nitric oxide concentration. This flow rate measurement also helps the system react more quickly to changes in flow rates, which can be common in ventilation circuits. Furthermore, the flow rate can help to determine a target sampling flow rate, such that the nitric oxide delivery apparatus is not drawing too much gas out of either the ventilation circuit or the ECMO sweep gas circuit for sampling.

The nitric oxide dosing cassettes 36004 and 36006 can be specifically targeted to ECMO treatment, wherein low sweep gas flow rates are common, such as flows from 0.5-3 liters per minute. In order to accommodate and accurately dose nitric oxide at these low flow rates, an ECMO specific cassette may contain a micro-orifice (as discussed and shown herein) for the delivery of nitric oxide which is sized explicitly for ECMO nitric oxide delivery flow rates. The micro-orifice may be less than 20 microns in diameter and may be from 1 micron to 20 microns, for example, 1 micron to 10 microns.

FIG. 39 contains a diagram of a setup according to some aspects, with a typical ECMO treatment setup involving an ECMO device as well as a ventilator. In the setup of the ECMO circuit, the patient's blood is pumped from the body 37040 through an ECMO device (oxygenator) 37042 that adds oxygen to the blood and removes carbon dioxide before being pumped back into the body (with blood rewarmed) 37044. The oxygenation and carbon dioxide removal are effected by the flow of a “sweep” gas 37046 provided by a blender 37048, which flows to the ECMO oxygenator 37042. This blender 37048 allows the user to control the mixture of air and oxygen, as well as the flow rate of the gas in the sweep gas 37046. In this sweep gas 37046, other gases or anesthetic agents may also be added. In some aspects, the system further includes a ventilator 37050 that provides breathing gas to the patient's lungs through mechanical breaths. The ventilator circuit includes an inspiratory gas path to the patient 37052 and an expiratory gas path 37054 from the patient back to the ventilator and/or an exhaust through an exhalation valve (not shown). In some aspects, the apparatus enables independent connections and delivery control of nitric oxide dosing into both the sweep gas 37046 of the ECMO circuit as well as into the inspiratory gas line 37052 to the patient on the ventilator. As with FIG. 38, FIG. 39 includes a nitric acid dosage system A that includes nitric oxide dosing cassette A 37004, nitric oxide dosing port 37010, and flow sensor port 37012 and a nitric acid dosage system B that includes nitric oxide dosing cassette B 37006, nitric oxide dosing port 37020, and flow sensor port 37022. As shown first in the ECMO circuit, nitric oxide is dosed 37056 to the sweep gas 37046. The flow rate of the sweep gas 37046 can be measured 37058 using flow sensor port 37012. In addition, the sweep gas 37046 can be sampled 37060 using gas sampling ports (shown generally as 37030) to determine the nitric oxide content, and optionally the content of other gases such as oxygen and nitrogen dioxide. Because the injection of nitric oxide into the ECMO device is typically achieved by injecting a mixture of air and nitric oxide from the blended 37048 into the ECMO circuit, there is some dilution of the oxygen in the sweep gas 37046 and, by measuring this, the apparatus can display the actual oxygen content of the diluted gas being supplied to the ECMO device on the graphical user interface (GUI) 37034. Furthermore, the apparatus can control the injection of nitric oxide to achieve a target O₂ dilution. In the ventilator circuit, nitric oxide is dosed 37062 to the inspiratory gas 37052. The flow rate of the inspiratory gas 37052 can be measured 37064 using flow sensor port 37022. In addition, the inspiratory gas 37052 can be sampled 37066 using gas sampling ports (shown generally as 37030) to determine the nitric oxide content, and optionally the content of other gases such as oxygen and nitrogen dioxide. In general, the GUI 37034 will display target and measured nitric oxide (in parts per million), oxygen %, and measured nitrogen dioxide (in parts per million) for each system A and B.

In some aspects, the apparatus can use nitric oxide dosing cassettes 37004 and 37006 can be targeted to larger patients. For example, in the case of a larger sweep gas 37046, the nitric oxide dosing cassette 37004 may contain provisions for larger outputs, such as a larger micro-orifice diameter. In these cases, the sweep gas flows may be higher, and the ventilator flow rates may either be high or low. The ability of the system to adapt to different flow rates allows the user to dose nitric oxide into the ECMO circuit and the ventilator circuit for patients from neonates to adults.

In some aspects, the nitric oxide dosing systems A and B in the apparatus have at least one control unit that is able to check and communicate with each dosing system and the system software that operates the GUI 37034 and external communications. The user can independently set nitric oxide dosing concentrations for the sweep gas 37046 and inspiratory gas 37052, and the system can display both of these values on the GUI 37034. Furthermore, in some embodiments, the system can provide a nitric oxide dosing index or value that represents the total amount of nitric oxide (the total nitric oxide dose) being delivered to the patient through both the ECMO circuit and the ventilator circuit. This nitric oxide dosing index or value can be presented as a single number that combines delivery concentrations and delivery flow rates and enables the user to quantify and view total nitric oxide delivery to the patient. For example, the nitric oxide dosing index or value can be determined using the following formula: ((Sweep gas flow rate x sweep gas nitric oxide concentration)+(ventilation gas flow rate times ventilation gas nitric oxide concentration)). This value can then be further scaled or non-dimensionalized for simplified presentation to a clinician/user as a nitric oxide dosing index. In some aspects, a user may target a total dose of nitric oxide to the patient, which can be automatically partitioned by the system between the sweep gas 37046 and the inspiratory gas 37052 and can furthermore be controlled based on target limits for each delivery circuit (i.e., the ECMO circuit and the ventilator circuit). The apparatus, through the use of software, can determine an optimal split of nitric oxide delivery between the sweep gas 37046 and the inspiratory gas 37052 and suggest this to the user for confirmation, or the apparatus can alternatively automatically control these values based on provided inputs from the user. In some aspects, the apparatus can include an electronic component that can communicate with a user through an external connection to a mobile device such as a smartphone. The system software can work with the electronic component to communicate with the mobile device through Bluetooth, Wi-Fi, or cellular communication, allowing the user to view the system's performance without needing to access the system console GUI 37034.

In some aspects, the apparatus can accept inputs from patient monitoring such as pulse oximetry (blood oxygen saturation), blood gas measurements (e.g., blood carbon dioxide saturation), and vital signs (e.g., pulse rate, temperature, respiration rate, blood pressure, and pH) which are used to assess the health of an ECMO patient. In this case, the system software can attempt to optimize, maximize, or minimize any of these patient parameters through the adjustment of nitric oxide dosing, including both the dosing levels as well as the split of delivery of nitric oxide between the sweep gas 37046 and the inspiratory gas 37052. This feedback loop would run separately from and provide inputs to the nitric oxide dose control feedback loops on each circuit (i.e., sweep gas 37046 and the inspiratory gas 37052).

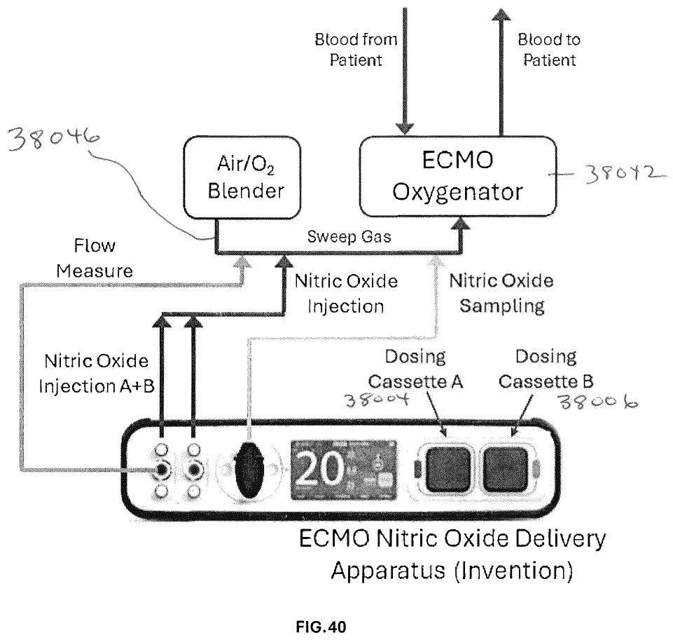

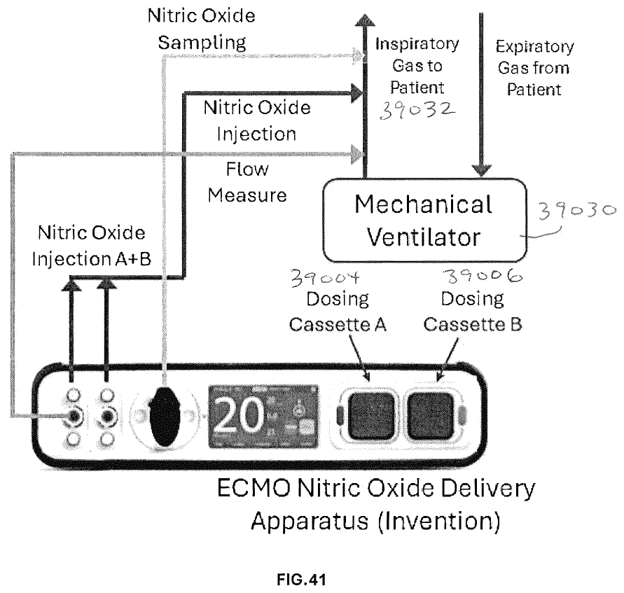

In some aspects, the system may accommodate a very large sweep gas flow for the ECMO circuit (FIG. 40) or a very large inspiratory gas flow for the ventilator circuit (FIG. 41) by utilizing both cassettes A and B to dose nitric oxide into the same circuit. For example, as shown in FIG. 40, the system can dose both the nitric oxide cassette A 38004 and the nitric oxide cassette B 38006 to the sweep gas 38046 for an ECMO device (oxygenator) 38042. Alternatively, as shown in FIG. 39, the system can dose both the nitric oxide cassette A 39004 and the nitric oxide cassette B 39006 to the inspiratory gas 39052 for a ventilator 39050. Because both dosing systems (cassettes) are controlled within the same apparatus and can communicate with each other, high parts per million dosing of nitric oxide (e.g., 80 ppm) into high flows in liters per minute (e.g., 70 liters per minute) can be provided to either the ECMO circuit or the ventilator circuit using a single system. In some aspects, a single high-dose ECMO cassette (e.g., 38004) may be capable of delivering high doses of nitric oxide into high flows of air plus oxygen.

In some aspects, the apparatus can be operated using an ECMO-specific mode or a CPB-specific mode (utilizing the ventilator). In this case, the apparatus would be capable of nitric oxide dosing into many other setups and scenarios, but the activation of the “ECMO Mode” or “CPB Mode” would specifically set the software and hardware configurations to the operations described. A user can activate the modes by pressing a button on the console or the GUI, or the apparatus can activate the mode by recognizing the insertion of an “ECMO” or “CPB” cassette. The apparatus can also read a barcode or RFID tag on the cassette or by other means of reading cassette data. Thus, the apparatus may either be completely directed to ECMO or ventilator usage, or it may have a mode which is ECMO-specific or CPB-specific (ventilator-specific).

While various aspects have been described above, it should be understood that they have been presented by way of example only and not limitation. Furthermore, although various aspects have been described as having particular features and/or combinations of components, other aspects possibly have a combination of any features and/or components from any of the aspects, where appropriate, as well as additional features and/or components.

Where methods described above indicate certain events occurring in a certain order, the ordering of certain events may be modified. Additionally, certain of the events may be performed concurrently in a parallel process, when possible, as well as performed sequentially as described above. Although various aspects have been described as having particular features and/or combinations of components, other aspects possibly have a combination of any features and/or components from any of the aspects, where appropriate.

### EXEMPLARY ASPECTS

Exemplary Aspect 1. A system comprising an apparatus for delivering a therapeutic amount of nitric oxide to a subject, the apparatus comprising at least one air inlet; a first receptacle configured to host a first source of nitric oxide; a second receptacle configured to host a second source of nitric oxide; a control unit configured to communicate with the first receptacle and the second receptacle and to selectively couple the at least one air inlet to the first receptacle and the second receptacle to deliver the nitric oxide from the first source and the second source; a first outlet coupled to the first receptacle and configured to deliver nitric oxide to a subject; and a second outlet coupled to the second receptacle and configured to deliver nitric oxide to a subject.

Exemplary Aspect 2. The system of any of the examples herein, particularly Exemplary Aspect 1, wherein the apparatus further comprises a graphical user interface (GUI) capable of displaying a nitric oxide content, a nitrogen dioxide content, and/or an oxygen content.

Exemplary Aspect 3. The system of any of the examples herein, particularly Exemplary Aspect 1 or 2, wherein the apparatus further comprises an electronic component for providing a nitric oxide content, a nitrogen dioxide content, and/or an oxygen content to a user's device.

Exemplary Aspect 4. The system of any of the examples herein, particularly Exemplary Aspects 1-3, wherein the first receptacle and/or the second receptacle has an orifice for delivering nitric oxide to the second outlet, the orifice having a diameter of no more than 20 microns.

Exemplary Aspect 5. The system of any of the examples herein, particularly Exemplary Aspect 4, wherein the orifice has a diameter of from 1 micron to 20 microns.

Exemplary Aspect 6. The system of any one of the previous any of the examples herein, particularly Exemplary Aspects 1-5, wherein the first outlet is in fluid communication with a ventilator and an extracorporeal membrane oxygen (ECMO) device.

Exemplary Aspect 7. The system of any one of the previous any of the examples herein, particularly Exemplary Aspects 1-6, wherein the second outlet is in fluid communication with a ventilator and an ECMO device.

Exemplary Aspect 8. The system of any of the examples herein, particularly Exemplary Aspect 6 or 7, wherein the ECMO device is further in fluid communication with a source of a flow of air, oxygen, or a combination thereof and the source of a flow of air, oxygen, or a combination thereof is combined with a flow from at least the second source of nitric oxide before entering the ECMO device.

Exemplary Aspect 9. The system of any of the examples herein, particularly Exemplary Aspects 6-8, wherein the control unit determines whether the first outlet delivers nitric oxide to a ventilator or an ECMO device, and whether the second outlet delivers nitric oxide to the ventilator or the ECMO device.

Exemplary Aspect 10. The system of any of the examples herein, particularly Exemplary Aspects 6-9, wherein the control unit controls whether the first outlet delivers nitric oxide to the ventilator or an ECMO device, and whether the second outlet delivers nitric oxide to the ventilator or the ECMO device.

Exemplary Aspect 11. The system of any of the examples herein, particularly Exemplary Aspects 1-10, wherein the control unit is configured to determine the total nitric oxide dose to be provided to a patient, for example, based on one or more of the following patient measurements: blood oxygen saturation, blood carbon dioxide saturation, pulse rate, temperature, respiration rate, blood pressure, and pH.

Exemplary Aspect 12. The system of any of the examples herein, particularly Exemplary Aspects 6-11, wherein the control unit is configured to determine, based on a first preset value of nitric oxide to be delivered in a first delivery flow to the ventilator, a first nitric oxide content to be provided in a first flow from the first outlet to provide the first preset value, for example, based on the amount of nitric oxide produced by the first source and the amount of air from the at least one air inlet directed to the first outlet.

Exemplary Aspect 13. The system of any of the examples herein, particularly Exemplary Aspect 12, wherein the control unit is configured to determine the first preset value of nitric oxide to be delivered in a first delivery flow to the ventilator.

Exemplary Aspect 14. The system of any of the examples herein, particularly Exemplary Aspects 6-13, wherein the control unit is configured to determine, based on a second preset value of nitric oxide to be delivered in a second delivery flow to the ECMO device, a second nitric oxide content to be provided in a second flow from the second outlet to provide the second preset value, for example, based on the amount of nitric oxide produced by the second source, the amount of air from the at least one air inlet directed to the second outlet, and the amount of additional air, oxygen, or a combination thereof being combined with the second flow.

Exemplary Aspect 15. The system of any of the examples herein, particularly Exemplary Aspect 14, wherein the control unit is configured to determine the second preset value of nitric oxide to be delivered in a second delivery flow to the ECMO device.

Exemplary Aspect 16. The system of any of the examples herein, particularly Exemplary Aspects 11-15, wherein the control unit is configured to determine, based on a total dose of nitric oxide to be provided to a patient, the first preset value of nitric oxide and the second preset value of nitric oxide.

Exemplary Aspect 17. The system of any of the examples herein, particularly Exemplary Aspects 1-16, wherein the apparatus further comprises at least one sampling port capable of measuring a nitric oxide content, a nitrogen dioxide content, and/or an oxygen content.

Exemplary Aspect 18. The system of any of the examples herein, particularly Exemplary Aspect 17, wherein a first delivery flow to a ventilator is in fluid communication with the at least one sampling port, and the control unit is configured to compare a first nitric oxide content in the first delivery flow to a first nitric oxide set value and to adjust the amount of nitric oxide delivered to the first outlet and provided to the ventilator.

Exemplary Aspect 19. The system of any of the examples herein, particularly Exemplary Aspect 17 or 18, wherein the control unit is configured to determine the first nitric oxide set value based on the first flow rate of a first delivery flow being delivered to the ventilator.

Exemplary Aspect 20. The system of any of the examples herein, particularly Exemplary Aspect 19, wherein the control unit is configured to adjust the first nitric oxide set value and/or the amount of air from the at least one air inlet being combined with the first source.

Exemplary Aspect 21. The system of any of the examples herein, particularly Exemplary Aspects 17-20, wherein a second delivery flow being delivered to the ECMO device is in fluid communication with the at least one sampling port, and the control unit is configured to compare a second nitric oxide content to a second nitric oxide set value and to adjust the amount of nitric oxide delivered to the second outlet and provided to the ECMO device.

Exemplary Aspect 22. The system of any of the examples herein, particularly Exemplary Aspect 20 or 21, wherein the control unit is configured to determine the second nitric oxide set value based on the second flow rate of a second delivery flow being delivered to the ECMO device.

Exemplary Aspect 23. The system of any of the examples herein, particularly Exemplary Aspect 22, wherein the control unit is configured to adjust the second nitric oxide set value and/or the amount of air from the at least one air inlet being combined with the second source.

Exemplary Aspect 24. A method for delivering a therapeutic amount of nitric oxide to a subject, comprising, providing an apparatus comprising: at least one air inlet; a first receptacle configured to host a first source of nitric oxide; a second receptacle configured to host a second source of nitric oxide; a control unit configured to selectively couple the at least one air inlet to the first receptacle and the second receptacle to deliver the nitric oxide from the first source and the second source; a first outlet coupled to the first receptacle and configured to deliver nitric oxide; and a second outlet coupled to the second receptacle and configured to deliver nitric oxide; and directing a first flow of nitric oxide and air, oxygen, or a combination thereof from the first outlet to a ventilator for delivery in a first delivery flow to a patient and/or directing a second flow of nitric oxide and air, oxygen, or a combination thereof from the second outlet to an extracorporeal membrane oxygen (ECMO) device in a second delivery flow for contact with the blood of the patient.

Exemplary Aspect 25. The method of any of the examples herein, particularly Exemplary Aspect 24, wherein the method comprises directing the first flow of nitric oxide and air, oxygen, or a combination thereof from the first outlet to the ventilator and directing the second flow of nitric oxide and air, oxygen, or a combination thereof from the second outlet to the ECMO device.

Exemplary Aspect 26. The method of any of the examples herein, particularly Exemplary Aspect 24 or 25, further comprising determining a total nitric oxide dose to be provided to a patient and determining whether the first outlet delivers nitric oxide to the ventilator or the ECMO device and determining whether the second outlet delivers nitric oxide to the ventilator or the ECMO device.

Exemplary Aspect 27. The method of any of the examples herein, particularly Exemplary Aspect 24 or 25, further comprising predetermining whether the first outlet delivers nitric oxide to the ventilator or the ECMO device and determining whether the second outlet delivers nitric oxide to the ventilator or the ECMO device.

Exemplary Aspect 28. The method of any one of any of the examples herein, particularly Exemplary Aspects 24-27, wherein the second flow is further combined with a blended flow of air, oxygen, or a combination thereof, and a second delivery flow being delivered to the ECMO device comprises the mixture of the second flow of nitric oxide and air, oxygen, or a combination thereof, and the blended flow of air, oxygen, or a combination thereof.

Exemplary Aspect 29. The method of any of the examples herein, particularly Exemplary Aspects 24-28, further comprising providing a first preset value of nitric oxide to be delivered in a first delivery flow to the ventilator for delivery to a patient and determining a first nitric oxide content to be provided in the first flow to provide the first preset value, for example, based on the amount of nitric oxide produced by the first source and the amount of air from the at least one air inlet directed to the first outlet.

Exemplary Aspect 30. The method of any of the examples herein, particularly Exemplary Aspects 24-29, further comprising providing a second preset value of nitric oxide to be delivered in a second delivery flow to the ECMO device and determining a first second nitric oxide content to be provided in the second flow to provide the second preset value, for example, based on the amount of nitric oxide produced by the second source, the amount of air from the at least one air inlet directed to the second outlet, and the amount of additional air, oxygen, or a combination thereof being combined with the second flow.

Exemplary Aspect 31. The method of any of the examples herein, particularly Exemplary Aspect 29 or 30, further comprising providing a total dose of nitric oxide to be provided to a patient and determining the first preset value of nitric oxide and the second preset value of nitric oxide.

Exemplary Aspect 32. The method of any of the examples herein, particularly Exemplary Aspect 31, further comprising determining the total dose of nitric oxide based on one or more of the following patient measurements: blood oxygen saturation, blood carbon dioxide saturation, pulse rate, temperature, respiration rate, blood pressure, and pH.

Exemplary Aspect 33. The method of any of the examples herein, particularly Exemplary Aspects 24-32, further comprising determining a nitric oxide dosing index using the following formula: (a first flow rate of the first delivery flow X a first nitric oxide concentration in the first delivery flow)+(a second flow rate of the second delivery flow X a second nitric oxide concentration in the second delivery flow).

Exemplary Aspect 34. The method of any of the examples herein, particularly Exemplary Aspects 24-33, further comprising sampling a first nitric oxide content in the first delivery flow, comparing the first nitric oxide content to a first nitric oxide set value and adjusting the amount of nitric oxide delivered to the first outlet based on the comparing step.

Exemplary Aspect 35. The method of any of the examples herein, particularly Exemplary Aspects 24-34, further comprising measuring a first flow rate of a first delivery flow being delivered to the ventilator and including the first flow of nitric oxide and air, oxygen, or a combination thereof and determining the first nitric oxide set value based on the first flow rate.

Exemplary Aspect 36. The method of any of the examples herein, particularly Exemplary Aspects 24-35, further comprising measuring a first flow rate of a first delivery flow being delivered to the ventilator and including the first flow of nitric oxide and air, oxygen, or a combination thereof and adjusting the first nitric oxide set value and/or the amount of air from the at least one air inlet being combined with the first source based on the first flow rate.

Exemplary Aspect 37. The method of any of the examples herein, particularly Exemplary Aspects 24-36, further comprising sampling a second nitric oxide content in the second delivery flow, comparing the second nitric oxide content to a second nitric oxide set value, and adjusting the amount of nitric oxide delivered to the second outlet based on the comparing step.

Exemplary Aspect 38. The method of any of the examples herein, particularly Exemplary Aspects 24-37, further comprising measuring a second flow rate of a second delivery flow being delivered to the ECMO device and including the second flow of nitric oxide and air, oxygen, or a combination thereof and determining the second nitric oxide set value based on the second flow rate.

Exemplary Aspect 39. The method of any of the examples herein, particularly Exemplary Aspects 24-38, further comprising measuring a second flow rate of a second delivery flow being delivered to the ECMO device and including the second flow of nitric oxide and air, oxygen, or a combination thereof and adjusting the second nitric oxide set value and/or the amount of air from the at least one air inlet being combined with the second source based on the second flow rate.

Exemplary Aspect 40. The method of any of the examples herein, particularly Exemplary Aspects 24-39, comprising a second flow rate of a second delivery flow being delivered to the ECMO device and including the second flow of nitric oxide and air, oxygen, or a combination thereof, wherein the second flow rate is from 0.5 to 3 liters per minute.

Exemplary Aspect 41. The method of any of the examples herein, particularly Exemplary Aspect 24, wherein the method comprises directing both the first flow of nitric oxide and air, oxygen, or a combination thereof from the first outlet and the second flow of nitric oxide and air, oxygen, or a combination thereof from the second outlet to the ventilator.

Exemplary Aspect 42. The method of any of the examples herein, particularly Exemplary Aspect 24, wherein the method comprises directing both the second flow of nitric oxide and air, oxygen, or a combination thereof from the second outlet and the first flow of nitric oxide and air, oxygen, or a combination thereof from the first outlet to the ECMO device.

What is claimed is:

1. A system comprising an apparatus for delivering a therapeutic amount of nitric oxide to a subject, the apparatus comprising:
at least one air inlet;
a first receptacle configured to host a first source of nitric oxide;
a second receptacle configured to host a second source of nitric oxide;
a control unit configured to communicate with the first receptacle and the second receptacle and to selectively couple the at least one air inlet to the first receptacle and the second receptacle to deliver the nitric oxide from the first source and the second source;
a first outlet coupled to the first receptacle and configured to deliver nitric oxide to a subject; and
a second outlet coupled to the second receptacle and configured to deliver nitric oxide to a subject.

2. The system of claim 1, wherein the first outlet is in fluid communication with a ventilator and an extracorporeal membrane oxygen (ECMO) device, and/or wherein the second outlet is in fluid communication with a ventilator and an ECMO device.

3. The system of claim 2, wherein the ECMO device is further in fluid communication with a source of a flow of air, oxygen, or a combination thereof and the source of a flow of air, oxygen, or a combination thereof is combined with a flow from at least the second source of nitric oxide before entering the ECMO device.

4. The system of claim 2, wherein the control unit determines whether the first outlet delivers nitric oxide to a ventilator or an ECMO device, and whether the second outlet delivers nitric oxide to the ventilator or the ECMO device, and/or wherein the control unit controls whether the first outlet delivers nitric oxide to the ventilator or an ECMO device, and whether the second outlet delivers nitric oxide to the ventilator or the ECMO device.

5. The system of 1, wherein the control unit is configured to determine the total nitric oxide dose to be provided to a patient based on one or more of the following patient measurements: blood oxygen saturation, blood carbon dioxide saturation, pulse rate, temperature, respiration rate, blood pressure, and pH.

6. The system of claim 2, wherein the control unit is configured:
a) to determine, based on a first preset value of nitric oxide to be delivered in a first delivery flow to the ventilator, a first nitric oxide content to be provided in a first flow from the first outlet to provide the first preset value based on the amount of nitric oxide produced by the first source and the amount of air from the at least one air inlet directed to the first outlet, and/or
b) to determine, based on a second preset value of nitric oxide to be delivered in a second delivery flow to the ECMO device, a second nitric oxide content to be provided in a second flow from the second outlet to provide the second preset value based on the amount of nitric oxide produced by the second source, the amount of air from the at least one air inlet directed to the second outlet, and the amount of additional air, oxygen, or a combination thereof being combined with the second flow.

7. The system of claim 6, wherein the control unit is configured to determine, based on a total dose of nitric oxide to be provided to a patient, the first preset value of nitric oxide and the second preset value of nitric oxide.

8. The system of claim 1, wherein the apparatus further comprises at least one sampling port capable of measuring a nitric oxide content, a nitrogen dioxide content, and/or an oxygen content.

9. The system of claim 8, wherein a first delivery flow to a ventilator is in fluid communication with the at least one sampling port, and the control unit is configured to compare a first nitric oxide content in the first delivery flow to a first nitric oxide set value and to adjust the amount of nitric oxide delivered to the first outlet and provided to the ventilator, and/or wherein a second delivery flow being delivered to the ECMO device is in fluid communication with the at least one sampling port, and the control unit is configured to compare a second nitric oxide content to a second nitric oxide set value and to adjust the amount of nitric oxide delivered to the second outlet and provided to the ECMO device.

10. The system of claim 9, wherein the control unit is configured to determine the first nitric oxide set value based on the first flow rate of a first delivery flow being delivered to the ventilator, and/or wherein the control unit is configured to determine the second nitric oxide set value based on the second flow rate of a second delivery flow being delivered to the ECMO device.

11. The system of claim 10, wherein the control unit is configured to adjust:
a) the first nitric oxide set value, and/or the amount of air from the at least one air inlet being combined with the first source, and/or
b) the second nitric oxide set value and/or the amount of air from the at least one air inlet being combined with the second source.

12. A method for delivering a therapeutic amount of nitric oxide to a subject, comprising,
providing an apparatus comprising:
at least one air inlet;
a first receptacle configured to host a first source of nitric oxide;
a second receptacle configured to host a second source of nitric oxide;
a control unit configured to selectively couple the at least one air inlet to the first receptacle and the second receptacle to deliver the nitric oxide from the first source and the second source;
a first outlet coupled to the first receptacle and configured to deliver nitric oxide; and
a second outlet coupled to the second receptacle and configured to deliver nitric oxide; and
directing a first flow of nitric oxide and air, oxygen, or a combination thereof from the first outlet to a ventilator for delivery in a first delivery flow to a patient and/or directing a second flow of nitric oxide and air, oxygen, or a combination thereof from the second outlet to an extracorporeal membrane oxygen (ECMO) device in a second delivery flow for contact with the blood of the patient.

13. The method of claim 12, wherein the method comprises directing the first flow of nitric oxide and air, oxygen, or a combination thereof from the first outlet to the ventilator and directing the second flow of nitric oxide and air, oxygen, or a combination thereof from the second outlet to the ECMO device.

14. The method of claim 12, further comprising:
a) determining a total nitric oxide dose to be provided to a patient and determining whether the first outlet delivers nitric oxide to the ventilator or the ECMO device and determining whether the second outlet delivers nitric oxide to the ventilator or the ECMO device or
b) predetermining whether the first outlet delivers nitric oxide to the ventilator or the ECMO device and determining whether the second outlet delivers nitric oxide to the ventilator or the ECMO device.

15. The method of claim 12, wherein the second flow is further combined with a blended flow of air, oxygen, or a combination thereof, and a second delivery flow being delivered to the ECMO device comprises the mixture of the second flow of nitric oxide and air, oxygen, or a combination thereof, and the blended flow of air, oxygen, or a combination thereof.

16. The method of claim 12, further comprising providing:
a) a first preset value of nitric oxide to be delivered in a first delivery flow to the ventilator for delivery to a patient and determining a first nitric oxide content to be provided in the first flow to provide the first preset value, based on the amount of nitric oxide produced by the first source and the amount of air from the at least one air inlet directed to the first outlet, and/or
b) a second preset value of nitric oxide to be delivered in a second delivery flow to the ECMO device and determining a first second nitric oxide content to be provided in the second flow to provide the second preset value, based on the amount of nitric oxide produced by the second source, the amount of air from the at least one air inlet directed to the second outlet, and the amount of additional air, oxygen, or a combination thereof being combined with the second flow.

17. The method of claim 16, further comprising:
a) providing a total dose of nitric oxide to be provided to a patient and determining the first preset value of nitric oxide and the second preset value of nitric oxide, and
b) determining the total dose of nitric oxide based on one or more of the following patient measurements: blood oxygen saturation, blood carbon dioxide saturation, pulse rate, temperature, respiration rate, blood pressure, and pH; and/or
c) determining a nitric oxide dosing index using the following formula: (a first flow rate of the first delivery flow X a first nitric oxide concentration in the first delivery flow)+(a second flow rate of the second delivery flow X a second nitric oxide concentration in the second delivery flow).

18. The method of claim 12, further comprising:
a) sampling a first nitric oxide content in the first delivery flow, comparing the first nitric oxide content to a first nitric oxide set value, and adjusting the amount of nitric oxide delivered to the first outlet based on the comparing step; and/or
b) measuring a first flow rate of a first delivery flow being delivered to the ventilator and including the first flow of nitric oxide and air, oxygen, or a combination thereof and determining the first nitric oxide set value based on the first flow rate; and/or
c) measuring a first flow rate of a first delivery flow being delivered to the ventilator and including the first flow of nitric oxide and air, oxygen, or a combination thereof and adjusting the first nitric oxide set value and/or the amount of air from the at least one air inlet being combined with the first source based on the first flow rate; and/or
d) sampling a second nitric oxide content in the second delivery flow, comparing the second nitric oxide content to a second nitric oxide set value, and adjusting the amount of nitric oxide delivered to the second outlet based on the comparing step; and/or
e) measuring a second flow rate of a second delivery flow being delivered to the ECMO device and including the second flow of nitric oxide and air, oxygen, or a combination thereof and determining the second nitric oxide set value based on the second flow rate; and/or
f) measuring a second flow rate of a second delivery flow being delivered to the ECMO device and including the second flow of nitric oxide and air, oxygen, or a combination thereof and adjusting the second nitric oxide set value and/or the amount of air from the at least one air inlet being combined with the second source based on the second flow rate.

19. The method of claim 12, comprising a second flow rate of a second delivery flow being delivered to the ECMO device and including the second flow of nitric oxide and air, oxygen, or a combination thereof, wherein the second flow rate is from 0.5 to 3 liters per minute.

20. The method of claim 12,
wherein the method comprises directing both the first flow of nitric oxide and air, oxygen, or a combination thereof from the first outlet and the second flow of nitric oxide and air, oxygen, or a combination thereof from the second outlet to the ventilator, or
wherein the method comprises directing both the second flow of nitric oxide and air, oxygen, or a combination thereof from the second outlet and the first flow of nitric oxide and air, oxygen, or a combination thereof from the first outlet to the ECMO device.
